(19) United States
(12) Patent Application Publication
(10) Pub. No.: US 2025/0120730 A1
(43) Pub. Date: Apr. 17, 2025

(54) ORTHOPEDIC IMPLANTS AND INSTRUMENTS

(71) Applicant: Paragon 28, Inc., Englewood, CO (US)

(72) Inventors: Darby MACLEISH, Denver, CO (US); Daniel J. LEE, Denver, CO (US); Joseph DOGUÉ, Aurora, CO (US); Mark Ray DALTON, Austin, TX (US)

(73) Assignee: Paragon 28, Inc., Englewood, CO (US)

(21) Appl. No.: 18/990,273

(22) Filed: Dec. 20, 2024

Related U.S. Application Data
(63) Continuation of application No. PCT/US2023/068893, filed on Jun. 22, 2023.
(60) Provisional application No. 63/366,817, filed on Jun. 22, 2022.

(51) Int. Cl. A61B 17/16; A61F 2/42

(52) U.S. Cl. CPC A61B 17/1682; A61F 2002/4205; A61F 2002/4207

(57) ABSTRACT

An orthopedic implant that includes a body. The body has a top surface opposite a bottom surface, a front surface opposite a rear surface, and a pair of lateral sides on opposing sides of the body from one another. At least a portion of the bottom surface of the body of the implant includes a concave geometry extending from one lateral side to the other lateral side, with the concave geometry biased such that the concave geometry is either closer to the front surface or the rear surface of the body of the implant. Also disclosed is an instrument configured to be coupled with an orthopedic trial. The instrument includes an engagement portion that attaches to other instruments that are configured to facilitate manipulation of the instrument. The engagement portion includes one or more apertures configured to receive at least a portion of one or more protrusions of an instrument.

### CROSS REFERENCE TO RELATED APPLICATION

This application is a bypass continuation of PCT Application No. PCT/US2023/068893, filed Jun. 22, 2023, and entitled “Orthopedic Implants and Instruments,” which claims benefit of priority of U.S. Provisional Patent Application No. 63/366,817, filed on Jun. 22, 2022, and entitled “Orthopedic Implants and Instruments” the disclosures of each of these applications are hereby incorporated herein by reference in their entirety.

### TECHNICAL FIELD

The present disclosure relates to implants, instruments, and corresponding methods for with performing orthopedic procedures. The present disclosure relates to podiatric and orthopedic implants and instruments that may be implemented in arthroplasty and/or other surgical procedures.

### BACKGROUND OF THE INVENTION

Many currently available implants, instruments, systems, and methods do not completely address the needs of patients. Additionally, many currently available implants, instruments, systems, and methods for ankle arthroplasty procedures fail to account for properties of joint anatomy and associated mechanical and kinematic movement patterns/capabilities.

### SUMMARY OF THE INVENTION

The present disclosure is directed toward an orthopedic implant. According to one aspect of the present disclosure, an orthopedic implant that includes a body. The body includes a top surface opposite a bottom surface, a front surface opposite a rear surface, and a pair of lateral sides opposite the body from one another. At least a portion of the bottom surface of the body of the implant includes a concave geometry extending from one lateral side to the other, with the concave geometry biased such that the concave geometry is either closer to the front surface or the rear surface of the body of the implant.

According to the first aspect of the present disclosure, the implant includes at least one aperture positioned on the front surface of the body of the implant.

According to the first aspect of the present disclosure, the at least one aperture includes a pair of apertures positioned on opposite sides of a midline of the implant.

According to the first aspect of the present disclosure, the concave geometry is biased such that the concave geometry is closer to the front surface of the body of the implant.

According to the first aspect of the present disclosure, the concave geometry is biased such that the concave geometry is closer to the rear surface of the body of the implant.

According to the first aspect of the present disclosure, the implant also includes a protrusion extending upward from the top surface of the body.

According to the first aspect of the present disclosure, the protrusion includes a substantially rectangular geometry.

According to the first aspect of the present disclosure, the protrusion includes a top surface that is spaced vertically from and in a plane that is substantially parallel to the top surface of the body of the implant.

According to the first aspect of the present disclosure, the protrusion includes a lateral dimension that increases along the height of the protrusion from adjacent the top surface of the body of the implant to the top surface of the protrusion.

According to the first aspect of the present disclosure, the protrusion includes a male dovetail component.

According to the first aspect of the present disclosure, the protrusion includes a notch extending upward from the top surface of the protrusion, wherein the notch is centered laterally about the protrusion.

According to the first aspect of the present disclosure, the implant is couplable with another implant via the protrusion and the notch.

A second aspect of the present disclosure is directed to an implant component. The implant component includes a body, which further includes a top surface opposite a bottom surface, a front surface opposite a rear surface, and a pair of lateral sides opposite the body from one another. At least a portion of the bottom surface of the body of the implant includes a concave geometry extending from one lateral side to the other and is positioned between the front and rear surfaces of the body.

According to the second aspect of the present disclosure, the concave geometry is positioned closer to the front surface of the body than the rear surface of the body.

According to the second aspect of the present disclosure, the concave geometry is positioned closer to the rear surface of the body than the front surface of the body.

According to the second aspect of the present disclosure, the implant component includes a protrusion extending upward from the top surface of the body of the implant component.

According to the second aspect of the present disclosure, the protrusion includes a top surface that is spaced vertically from and in a plane substantially parallel to the top surface of the body of the implant.

According to the second aspect of the present disclosure, the protrusion includes a lateral dimension that increases along the height of the protrusion from adjacent the top surface of the body of the implant to the top surface of the protrusion.

According to the second aspect of the present disclosure, the protrusion includes a male dovetail component. A third aspect of the present disclosure is directed to an orthopedic implant. The implant includes a body, which further includes a top surface opposite a bottom surface, a front surface opposite a rear surface, and a pair of lateral sides opposite the body from one another. At least a portion of the bottom surface of the body of the implant includes a concave geometry extending from one lateral side to the other, with the concave geometry biased such that the concave geometry is either closer to the front surface or the rear surface of the body of the implant. The implant also includes a protrusion extending upward from the top surface of the body and having a top surface in a plane substantially parallel to the top surface of the body, wherein the protrusion includes a lateral dimension that increases along the height of the protrusion from adjacent the top surface of the body to the top surface of the protrusion.

A fourth aspect of the present disclosure is directed to an instrument configured to be releasably coupled with an orthopedic trial. The instrument includes an engagement portion configured to releasably couple with one or more other instruments configured to facilitate manipulation, positioning, and/or repositioning of the instrument. The engagement portion includes one or more holes or apertures configured to receive at least a portion of one or more protrusions of an instrument to facilitate releasable coupling.

A fifth aspect of the present disclosure is directed to an instrument. The instrument includes a handle and an actuator positioned adjacent the handle, with the instrument further including a resilient member disposed adjacent the handle and between two portions of the handle such that the resilient member may bias the two portions of the handle apart from one another. The actuator may be actuated to retain the two portions of the handle in a desired position. The instrument also includes a four-bar mechanism coupled with the handle, and a punch releasably coupled to a portion of the four-bar mechanism. The punch includes an upper portion configured to receive at least a portion of the four-bar mechanism therein to facilitate releasable coupling therewith, and a platform opposite the upper portion from the four-bar mechanism, with the platform integral with the upper portion. The punch further includes a pair of projections positioned at a terminal end of the platform, wherein the pair of projections extend in an upward and substantially perpendicular direction from the platform.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

FIG. 1 is a front perspective view of an orthopedic trial, in accordance with the present disclosure;

FIG. 2 is a front view of the orthopedic trial of FIG. 1, in accordance with the present disclosure;

FIG. 3 is a rear view of the orthopedic trial of FIG. 1, in accordance with the present disclosure;

FIG. 4 is a first side view of the orthopedic trial of FIG. 1, in accordance with the present disclosure;

FIG. 5 is a second side view of the orthopedic trial of FIG. 1, in accordance with the present disclosure;

FIG. 6 is a top view of the orthopedic trial of FIG. 1, in accordance with the present disclosure;

FIG. 7 is a bottom view of the orthopedic trial of FIG. 1, in accordance with the present disclosure;

FIG. 8 is a front perspective view of the orthopedic trial of FIG. 1 shown in conjunction with at least a portion of an implant system, in accordance with the present disclosure;

FIG. 9 is a first side view of the orthopedic trial of FIG. 1 in conjunction with at least a portion of the implant system of FIG. 8, in accordance with the present disclosure;

FIG. 10 is a rear view of the orthopedic trial of FIG. 1 in conjunction with at least a portion of the implant system of FIG. 8, in accordance with the present disclosure;

FIG. 11 is a second side view of the orthopedic trial of FIG. 1 in conjunction with at least a portion of the implant system of FIG. 8, in accordance with the present disclosure;

FIG. 12 is a front view of the orthopedic trial of FIG. 1 in conjunction with at least a portion of the implant system of FIG. 8, in accordance with the present disclosure;

FIG. 13 is a front perspective view of an alternate configuration of the orthopedic trial of FIG. 1 in conjunction with at least a portion of the implant system of FIG. 8, in accordance with the present disclosure;

FIG. 14 is a first side view of the orthopedic trial of FIG. 1 (in the configuration of FIG. 13) in conjunction with at least a portion of the implant system of FIG. 8, in accordance with the present disclosure;

FIG. 15 is a second side view of the orthopedic trial of FIG. 1 (in the configuration of FIG. 13) in conjunction with at least a portion of the implant system of FIG. 8, in accordance with the present disclosure;

FIG. 16 is a rear view of the orthopedic trial of FIG. 1 (in the configuration of FIG. 13) in conjunction with at least a portion of the implant system of FIG. 8, in accordance with the present disclosure;

FIG. 17 is a front view of the orthopedic trial of FIG. 1 (in the configuration of FIG. 13) in conjunction with at least a portion of the implant system of FIG. 8, in accordance with the present disclosure;

FIG. 18 is a front perspective view of the orthopedic trial of FIG. 1 in conjunction with an alternate orthopedic instrument, in accordance with the present disclosure;

FIG. 19 is a first side view of the orthopedic trial of FIG. 1 in conjunction with the orthopedic instrument of FIG. 18, in accordance with the present disclosure;

FIG. 20 is a second side view of the orthopedic trial of FIG. 1 in conjunction with the orthopedic instrument of FIG. 18, in accordance with the present disclosure;

FIG. 21 is a front view of the orthopedic trial of FIG. 1 in conjunction with the orthopedic instrument of FIG. 18, in accordance with the present disclosure;

FIG. 22 is a front perspective view of the orthopedic instrument of FIG. 1 (in an alternate configuration) in conjunction with the orthopedic instrument of FIG. 18, in accordance with the present disclosure;

FIG. 23 is a first side view of the orthopedic trial of FIG. 1 (in the alternate configuration of FIG. 22) in conjunction with the orthopedic instrument of FIG. 18, in accordance with the present disclosure;

FIG. 24 is a second side view of the orthopedic trial of FIG. 1 (in the alternate configuration of FIG. 22) in conjunction with the orthopedic instrument of FIG. 18, in accordance with the present disclosure;

FIG. 25 is a front view of the orthopedic trial of FIG. 1 (in the alternate configuration of FIG. 22) in conjunction with the orthopedic instrument of FIG. 18, in accordance with the present disclosure;

FIG. 26 is a rear view of the orthopedic trial of FIG. 1 (in the alternate configuration of FIG. 22) in conjunction with the orthopedic instrument of FIG. 18, in accordance with the present disclosure;

FIG. 27 is a perspective view of an orthopedic instrument, in accordance with the present disclosure;

FIG. 28 is a side perspective view the orthopedic instrument of FIG. 27, in accordance with the present disclosure;

FIG. 29 is an enlarged side perspective view of a portion of the orthopedic instrument of FIG. 27, in accordance with the present disclosure;

FIG. 30 is a front view of the portion of the orthopedic instrument of FIG. 29, in accordance with the present disclosure;

FIG. 31 is a first side view of the portion of the orthopedic instrument of FIG. 29, in accordance with the present disclosure;

FIG. 32 is a second side view of the portion of the orthopedic instrument of FIG. 29, in accordance with the present disclosure;

FIG. 33 is a bottom perspective view of the portion of the orthopedic instrument of FIG. 29, in accordance with the present disclosure;

FIG. 34 is a side perspective view of another orthopedic instrument, in accordance with the present disclosure;

FIG. 35 is a top perspective view of the orthopedic instrument of FIG. 34, in accordance with the present disclosure;

FIG. 36 is a rear perspective view of another orthopedic instrument, in accordance with the present disclosure;

FIG. 37 is a side perspective view of the orthopedic instrument of FIG. 36, in accordance with the present disclosure;

FIG. 38 is a front view of the orthopedic instrument of FIG. 36, in accordance with the present disclosure;

FIG. 39 is a front perspective view of an orthopedic implant, in accordance with the present disclosure;

FIG. 40 is a front view of the orthopedic implant of FIG. 39, in accordance with the present disclosure;

FIG. 41 is a rear view of the orthopedic implant of FIG. 39, in accordance with the present disclosure;

FIG. 42 is a first side view of the orthopedic implant of FIG. 39, in accordance with the present disclosure;

FIG. 43 is a second side view of the orthopedic implant of FIG. 39, in accordance with the present disclosure;

FIG. 44 is a top view of the orthopedic implant of FIG. 39, in accordance with the present disclosure;

FIG. 45 is a bottom view of the orthopedic implant of FIG. 39, in accordance with the present disclosure;

FIG. 46 is a front perspective view of the orthopedic implant of FIG. 39 in conjunction with the implant system of FIG. 8, in accordance with the present disclosure;

FIG. 47 is a front view of the orthopedic implant of FIG. 39 in conjunction with the implant system of FIG. 8, in accordance with the present disclosure;

FIG. 48 is a first side view of the orthopedic implant of FIG. 39 in conjunction with the implant system of FIG. 8, in accordance with the present disclosure;

FIG. 49 is a second side view of the orthopedic implant of FIG. 39 in conjunction with the implant system of FIG. 8, in accordance with the present disclosure;

FIG. 50 is a front view of alternative orthopedic implant, in accordance with the present disclosure;

FIG. 51 is a rear view of the orthopedic implant of FIG. 50, in accordance with the present disclosure;

FIG. 52 is a first side view of the orthopedic implant of FIG. 50, in accordance with the present disclosure;

FIG. 53 is a second side view of the orthopedic implant of FIG. 50, in accordance with the present disclosure;

FIG. 54 is a top view of the orthopedic implant of FIG. 50, in accordance with the present disclosure;

FIG. 55 is a bottom view of the orthopedic implant of FIG. 50, in accordance with the present disclosure;

FIG. 56 is a front perspective view of the orthopedic implant of FIG. 50 in conjunction with the implant system of FIG. 8, in accordance with the present disclosure;

FIG. 57 is a front view of the orthopedic implant of FIG. 50 in conjunction with the implant system of FIG. 8, in accordance with the present disclosure;

FIG. 58 is a first side view of the orthopedic implant of FIG. 50 in conjunction with the implant system of FIG. 8, in accordance with the present disclosure;

FIG. 59 is a second side view of the orthopedic implant of FIG. 50 in conjunction with the implant system of FIG. 8, in accordance with the present disclosure;

FIG. 60 is a side perspective view of an orthopedic instrument, in accordance with the present disclosure;

FIG. 61 is a top perspective view of the orthopedic instrument of FIG. 60, in accordance with the present disclosure;

FIG. 62 is an alternate perspective view of the orthopedic instrument of FIG. 60, in accordance with the present disclosure;

FIG. 63 is a side perspective view of an orthopedic instrument, in accordance with the present disclosure;

FIG. 64 is a top perspective view of the orthopedic instrument of FIG. 63, in accordance with the present disclosure; and

FIG. 65 is a side view of the orthopedic instrument of FIG. 63, in accordance with the present disclosure.

### DETAILED DESCRIPTION OF THE INVENTION

In this detailed description and the following claims, the words proximal, distal, anterior, or plantar, posterior, or dorsal, medial, lateral, superior, and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers the bottom of the foot.

Similarly, positions or directions may be used herein with reference to anatomical structures or surfaces. For example, as the current implants, devices, instrumentation, and methods are described herein with reference to use with the bones of the foot, the bones of the foot, ankle and lower leg may be used to describe the surfaces, positions, directions or orientations of the implants, devices, instrumentation, and methods. Further, the implants, devices, instrumentation, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation, and methods, and the aspects, components, features and the like thereof, described herein with respect to the right foot may be mirrored so that they likewise function with the left foot. Further, the implants, devices, instrumentation, and methods, and the aspects, components, features and the like thereof, disclosed herein are described with respect to the foot for brevity purposes, but it should be understood that the implants, devices, instrumentation, and methods may be used with other bones of the body having similar structures.

This application is cross-related to U.S. application Ser. No. 17/647,646 filed on Jan. 11, 2022, which is a continuation of U.S. application Ser. No. 16/949,310 filed Oct. 24, 2020, which issued as U.S. Pat. No. 11,219,530 on Jan. 11, 2022, which is a National Stage application based on International Application No. PCT/US2019/29009 filed on Apr. 24, 2019 and published as WO 2020/013901, which claims priority benefit under 35 U.S.C. § 119(e) to U.S. provisional application No. 62/661,945 filed Apr. 24, 2019 which are each incorporated herein by reference in their entireties.

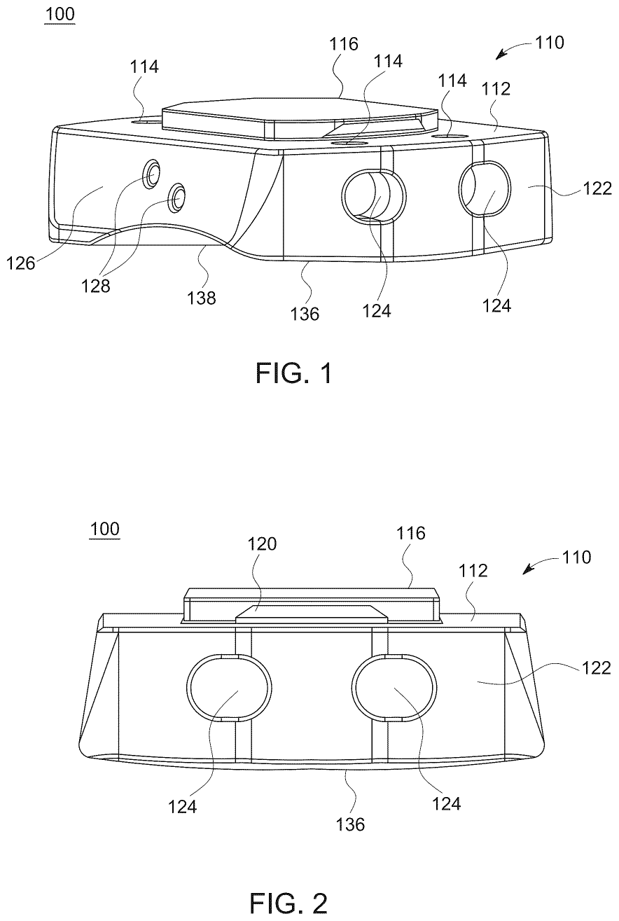

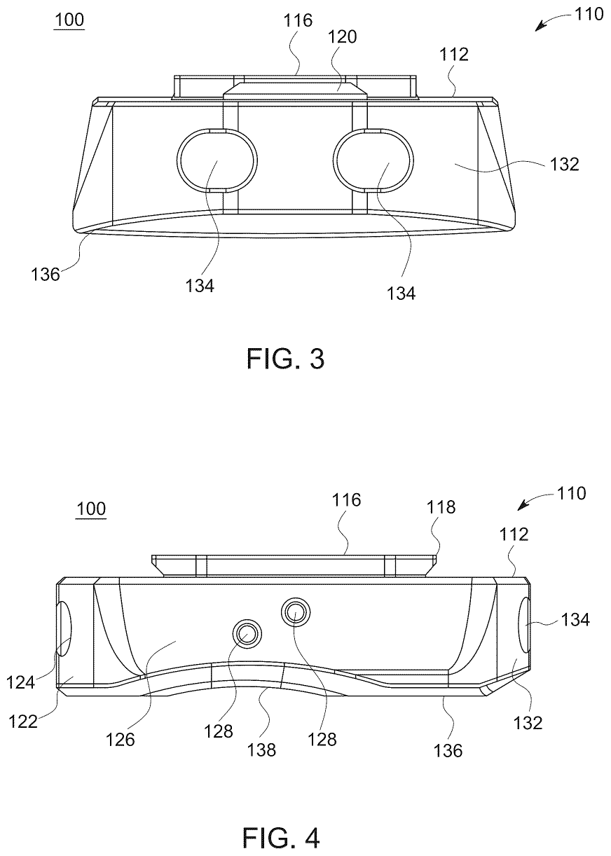

Referring now to FIGS. 1-7, an orthopedic trial 100 is shown, according to one aspect of the present disclosure. In some aspects, the trial 100 may be a component of a kit or system (e.g., an implant kit or implant system) where the kit or system includes one or more of the trials 100, for example in various geometries and/or sizes. Further, in some aspects the trial 100 may correspond to one or more implant or instrument components of a kit or system as mentioned previously, where the trial 100 may include one or more geometries and/or features configured to interface with one or more components of such a kit or system (e.g., a feature to accommodate releasable coupling with an instrument, a contour configured to interface with an implant component, etc.).

The trial 100 is shown to include a body 110 having an upper surface 112 opposite a lower surface 136, a pair of lateral surfaces 126 opposite from one another, and a front surface 122 opposite the rear surface 132. As shown, the body 110 has a substantially rectangular prism-based configuration, where the upper and lower surfaces 112, 136, the lateral surfaces 126, and the front and rear surfaces 122, 132 are substantially perpendicular to one another, respectively. The body 110 is shown to include a protrusion 116 extending in an upward direction from the upper surface 112, where the protrusion includes a ledge 118 extending at least partially around the perimeter of the protrusion 116 between the upper surface 112 of the body 110 and a top surface of the protrusion 116 which is substantially parallel to the upper surface 112. In some aspects, the protrusion 116 may be configured to include a substantially octagonal or rectangular prism-based geometry or other similar and/or alternate geometries. In some aspects, the protrusion 116 and the ledge 118 thereof may collectively form a coupling means, for example to facilitate releasable coupling with one or more other instruments and/or implant components. In some aspects, the trial 100 may be configured to releasably couple with instruments and/or implants via the protrusion 116 and the ledge 118 in a dovetail configuration, where the protrusion 116 and the ledge 118 form a male dovetail component and are received in the instruments and/or implants by a female dovetail component. As shown, the protrusion 116 includes a notch 120 positioned between the protrusion 116 and the front surface 122 of the trial 100, with the notch 120 being centered relative to the protrusion 116 (with the protrusion 116 also being centered relative to the top surface 112 of the trial 100). In some aspects, the top surface 112 may also include one or more apertures 114 positioned about the top surface 112 and extending at least partially into the body 110 of the trial 100 in a direction toward the lower surface 136. In some aspects, the apertures 114 may be configured to releasably couple the trial 100 with an instrument, for example an insertion instrument configured to facilitate positioning the trial 100 in a desired location as part of an implant trialing/size determination process, or other components of an implant/instrument kit/system. As shown, the trial 100 includes four apertures, with two positioned between the protrusion 116 and the front surface 122 and two positioned between the protrusion 116 and the rear surface 132 (although the one or more apertures may be positioned in various configurations in some aspects).

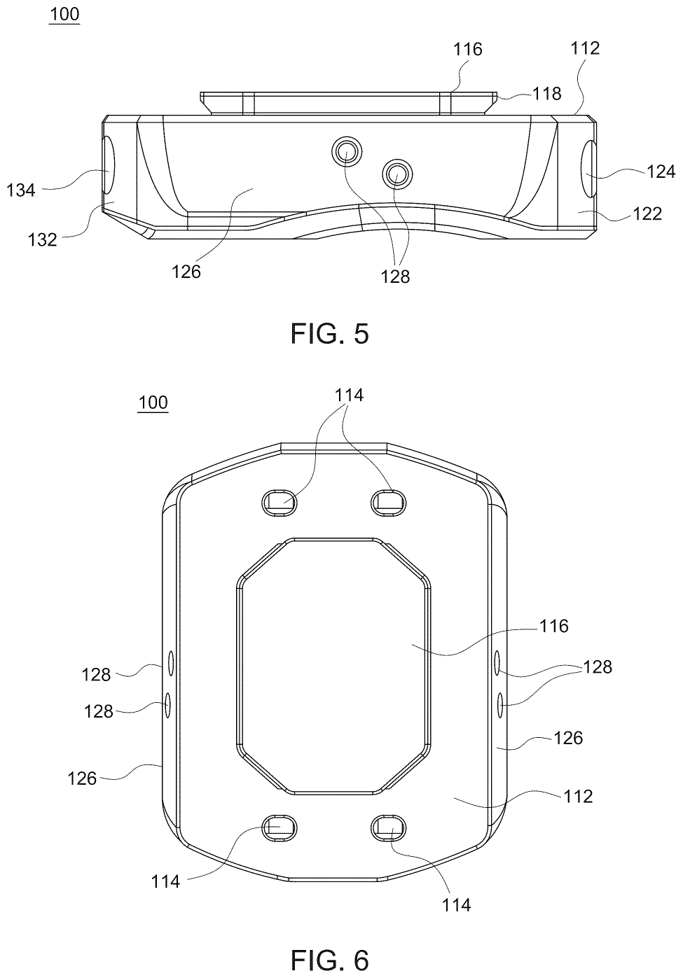

The front surface 122 and the rear surface 132 (which are marked with “A” and “P” to indicate “anterior” and “posterior”, respectively, where the front surface 122 may be referred to as the anterior surface 122 and the rear surface 132 may be referred to as the posterior surface 122) are each shown to include a pair of apertures 124, 134, respectively, disposed on the surfaces and extending into the body 110 of the trial 100 in a direction substantially perpendicular to planes in which the front and rear surfaces 122, 132 are positioned. In some aspects, the apertures 124, 134 may be configured to releasably couple the trial 100 with an instrument, for example an insertion instrument configured to facilitate positioning the trial 100 in a desired location as part of an implant trialing/size determination process, or other components of an implant/instrument kit/system. For example, one or more of the apertures 124, 134 may be configured to receive a protrusion of an instrument and retain at least a portion of the protrusion therein while the trial 100 is manipulated, positioned, and/or repositioned. The lateral sides 126 of the trial 100 are also shown to each include a pair of apertures 128, with the apertures 128 being positioned symmetrically about a midline of the trial 100 when viewed from a top view such as that of FIG. 6. In some aspects, the apertures 128 may be configured to provide fluid communication between the pair of lateral sides 126 (e.g., extend through trial). Further, in some aspects the apertures 128 may include one or more radiopaque elements disposed therein such that the radiopaque elements may be easily viewed under fluoroscopy in contrast to the radiolucent body 110. In some aspects, each pair of apertures 128 (e.g., the pair of apertures 128 on a single lateral side 126) may be staggered relative to one another in multiple directions. For example, as shown in FIG. 5, one aperture 128 may be nearer the upper and/or rear surfaces 112, 132 than the other aperture 128 (which would accordingly be nearer the front and lower surfaces 122, 136).

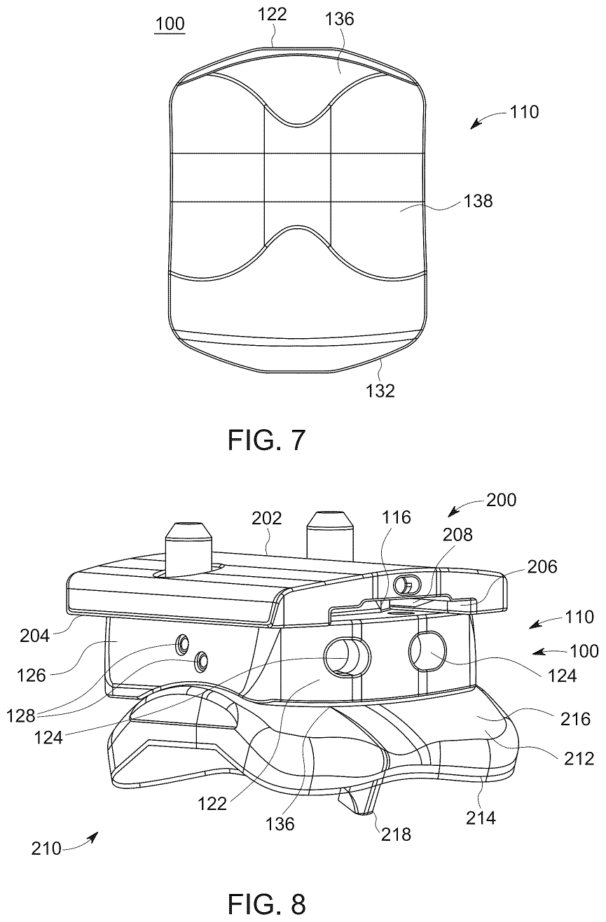

The trial 100 is further shown to include a concavity 138 positioned on/within at least a portion of the lower surface 136. In some aspects, the concavity 138 may include a ridge along a central portion thereof separating two smaller sub-concavities. Further, in some aspects the concavity 138 may correspond to a geometry of one or more implants and/or portions thereof with which the trial 100 is configured to interface in a trialing/size determining process. For example, the concavity 138 may correspond to a convexity/curvature of an upper surface of a talar implant with which the trial 100 is configured to interface. As shown in FIG. 4, the concavity 138 is biased toward the front surface 122 of the trial 100 (e.g., anteriorly, “left-biased” in FIG. 4). However, when the trial 100 is reversed (e.g., FIG. 5) the concavity 138 is biased in the opposite direction (e.g., “right-biased”). Accordingly, the trial 100 may be inserted such that the front surface 122 is facing a user to facilitate trialing for an anteriorly-biased implant, and conversely may be inserted such that the rear face 132 is facing a user to facilitate for a posteriorly-biased implant. That is to say that the trial 110 may be used reversibly, and thus is compatible for trialing/size determining processes for both anteriorly and posteriorly-biased implants. Accordingly, the trial 100 (and/or components/features thereof) (as shown in at least FIGS. 1-7) has at least one line of symmetry, for example, a line of symmetry running vertically through the trial as oriented in FIGS. 6 and 7.

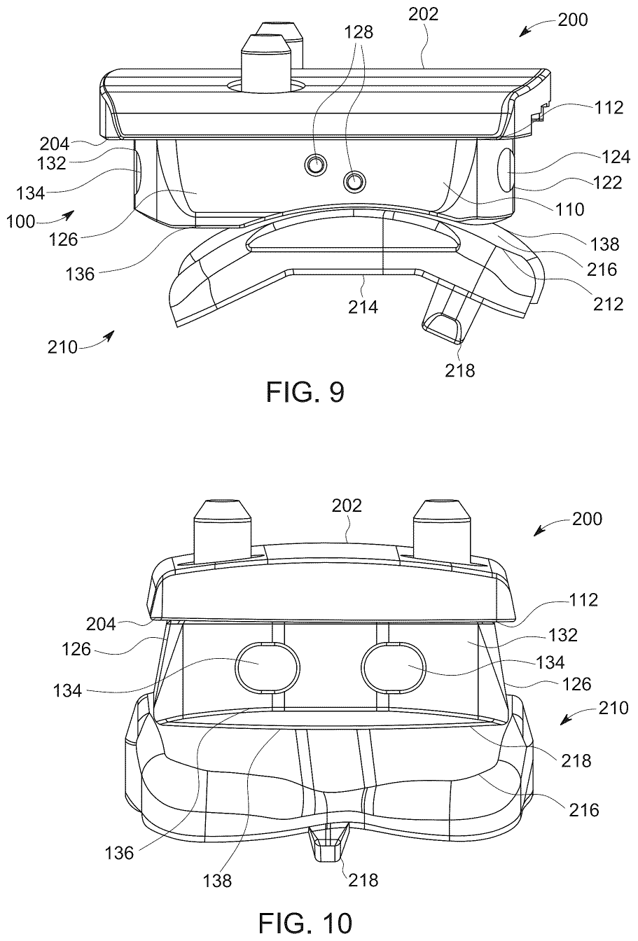

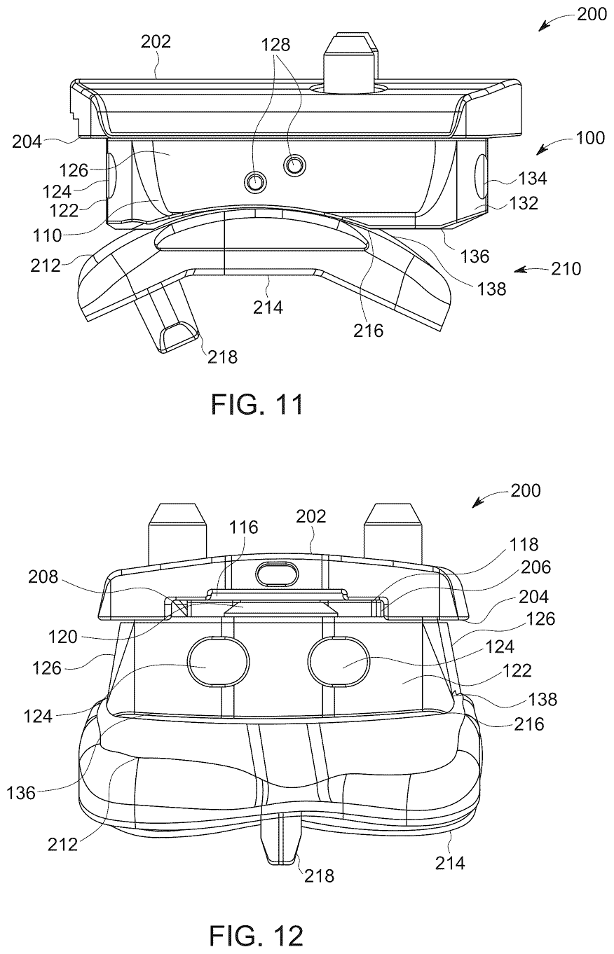

Referring now to FIGS. 8-12, the trial 100 is shown in conjunction with an implant system including a tibial component 200 and a talar component 210. The tibial and talar components 200, 210 may be the same as and/or similar to other similar tibial and talar components of ankle arthroplasty systems including but not limited to those incorporated by reference herein. As shown, the tibial component 200 includes a top surface 202 opposite a bottom surface 204, with the bottom surface including a recess 206 extending into the bottom surface 204 towards the top surface 202. The top surface 202 is shown to include at least one protrusion (two as shown in FIGS. 8-12) configured to facilitate fixation to/in the distal tibia of a patient. The recess 206 is configured to include an engagement feature 208 disposed therein which, as shown in at least FIGS. 8-12, includes a female dovetail configuration configured to interface and releasably couple with the protrusion 116, the ledge 118 and/or the notch 120 of the trial 100, thus releasably coupling the trial 100 with the tibial component 200. In some aspects, the trial 100 may be configured to engage/releasably couple with the tibial component 210 in the same manner as an implant component for which the trial 100 corresponds (e.g., an intermediate implant component may include the same/similar features to couple with the tibial component 200 in the same/similar manner). When engaged (e.g., releasably coupled) with the tibial component 200, the trial 100 is shown to be positioned substantially below (e.g., inferior, distal, etc.) relative to the tibial component).

The talar component 210 is shown to be positioned such that the trial 100 is disposed substantially above (e.g., superior, proximal, etc.) the talar component 210. The talar component 210 is shown to include a top surface 212 positioned substantially opposite the talar component 210 from a bottom surface 214, where the top surface 212 is configured to engage/interface with the trial 100 and the bottom 214 surface includes at least one engagement feature 218 configured to facilitate coupling with a resected bone surface of the talus of a patient. The top surface 212 of the talar component 210 is shown to include an articulation 216, which may be the same as and/or similar to that shown and described in the aforementioned issued patents/pending applications that are incorporated herein by reference. For example, the articulation 216 may include the corresponding articulating surface being broken into four regions for reference, with the regions being anterior-medial, anterior-lateral, posterior-medial, and posterior-lateral (where the anterior portion of the talar component 210 is the portion that includes the engagement feature 218), with each region having at least one sagittal radius. In some aspects, the anterior-lateral sagittal radius may be greater than the anterior-medial sagittal radius, and the posterior-medial sagittal radius may be greater than the posterior-lateral sagittal radius. The concavity 138 of the trial 100 is configured to interface with the articulation 216 and, accordingly, may have a geometry that is complimentary to (e.g., a negative of) the articulation 216 and substantially the same and/or similar to a concavity of an implant component of which the trial 100 is representative.

As shown in FIGS. 8-12, the trial 100 is shown (see at least the front view of FIG. 12) with the front surface 122 facing outward and adjacent the front (e.g., anterior) portions of the tibial and talar components 200, 210, with the “A” of the trial 100 indicating that the concavity 138 is biased anteriorly (e.g., closer to the front/anterior surface 122). Accordingly, trialing/size determination processes performed with the trial 100 in such a configuration will allow for a physician to determine a desired implant size (based on the fit of various different sizes of the trial 100, each with the same/similar features as those shown and described herein) for an anteriorly-biased implant (e.g., an implant that will have a concavity 138 configured to interface with the articulation 216 of the talar component 210 and positioned closer to the anterior/front surface 122 than posterior/rear surface 132).

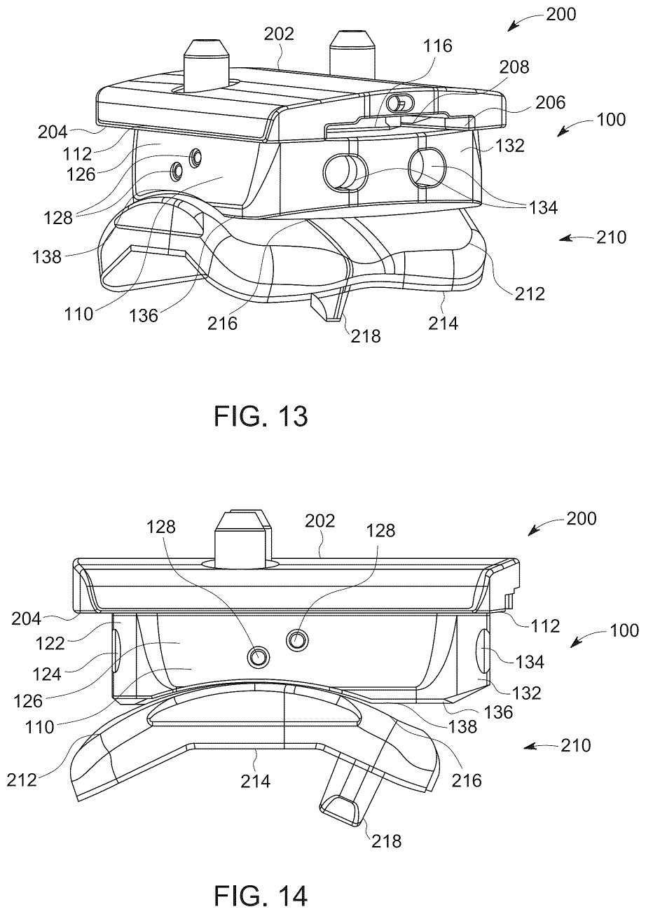

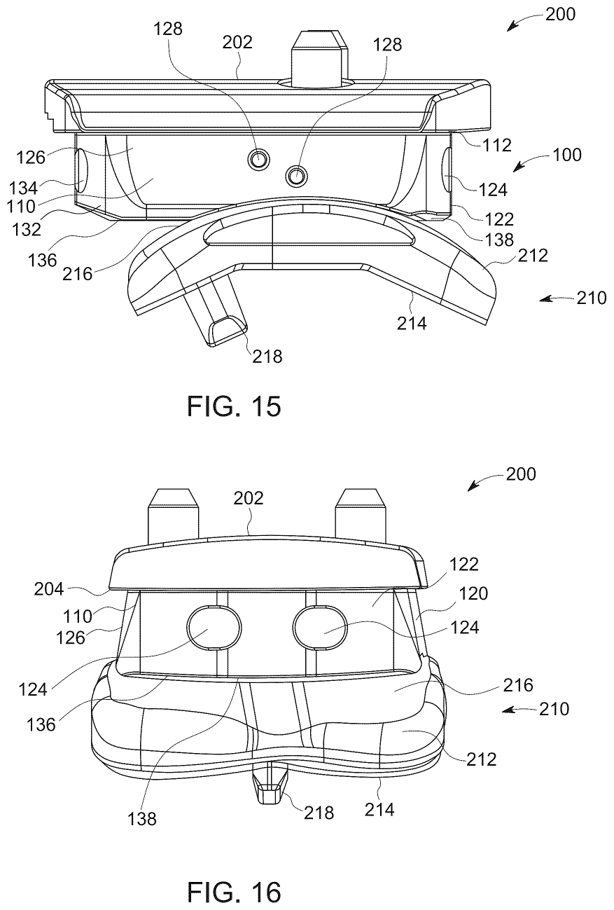

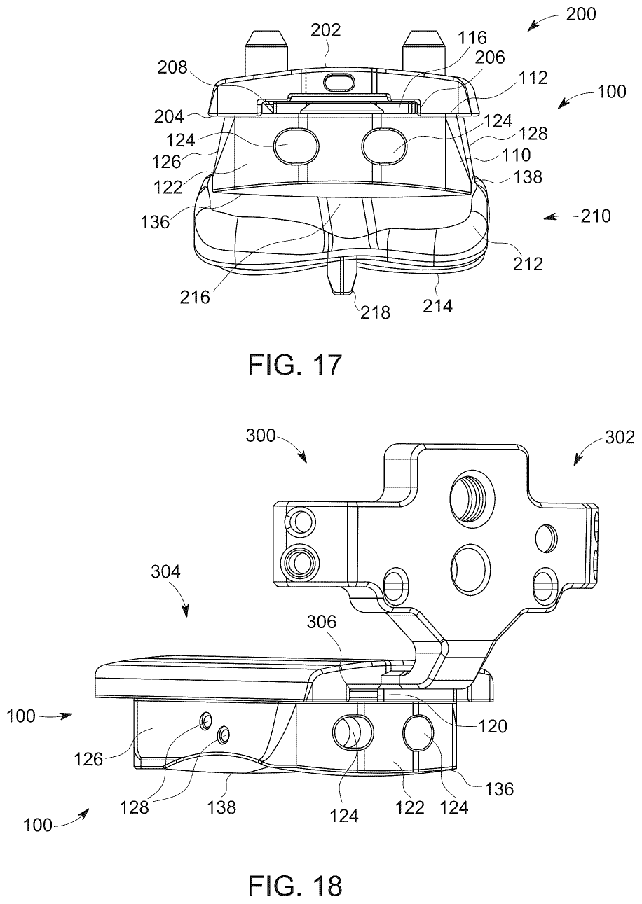

Referring now to FIGS. 13-17, the same components as shown in FIGS. 8-12 are shown, but in an alternate configuration. The trial 100 is shown (see at least the front view of FIG. 17) with the rear surface 132 facing outward and adjacent the front (e.g., anterior) portions of the tibial and talar components 200, 210, with the “P” of the trial 100 indicating that the concavity 138 is biased posteriorly (e.g., closer to the rear/posterior surface 132). Accordingly, the trialing/size determination processes performed with the trial 100 in such a configuration will allow for a physician to determine a desired implant size (based on the fit of the various different sizes of the trial 100, each with the same/similar features as those shown and described herein) for a posteriorly-biased implant (e.g., an implant that will have a concavity 138 configured to interface with the articulation 216 of the talar component 210 and positioned closer to the posterior/rear surface 132 than anterior/front surface 122).

When implementing the trial 100 as shown and described previously herein with reference to FIGS. 8-12 (anteriorly-biased configuration) and FIGS. 13-17 (posteriorly-biased configuration), a physician may perform a trialing process for both anterior and posterior-biased implant components using the trial 100 (or multiple sizes of the trial 100 provided in a system/kit, with each size of the trial 100 having the same/similar features to those shown and described herein). For example, a physician may desire an anteriorly-biased implant for a patient and, accordingly, perform a trialing process with the trial 100 in the position as shown in FIGS. 8-12. However, if during the trialing process for an anteriorly-biased implant the physician determines that a posteriorly-biased implant better suits the patient, the physician may perform the corresponding trialing process for the posteriorly-biased implant using the same trial 100, but in the reverse (e.g., “flipped”) configuration (e.g., transition from the front/anterior surface 122 of the trial 100 being visible and facing the physician/an anterior direction when placed to the rear/posterior surface 132 of the trial 100 being visible and facing the physician/an anterior direction).

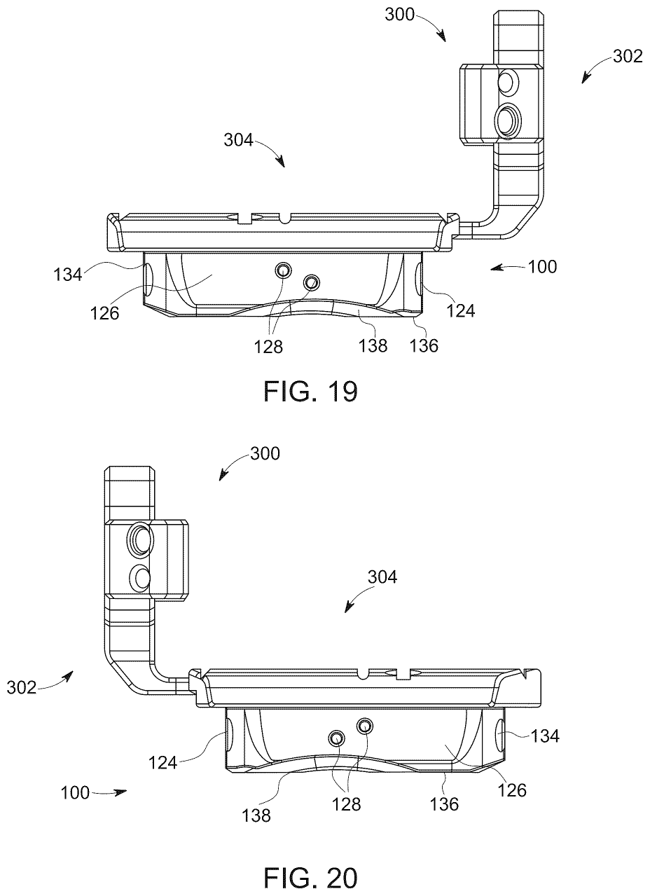

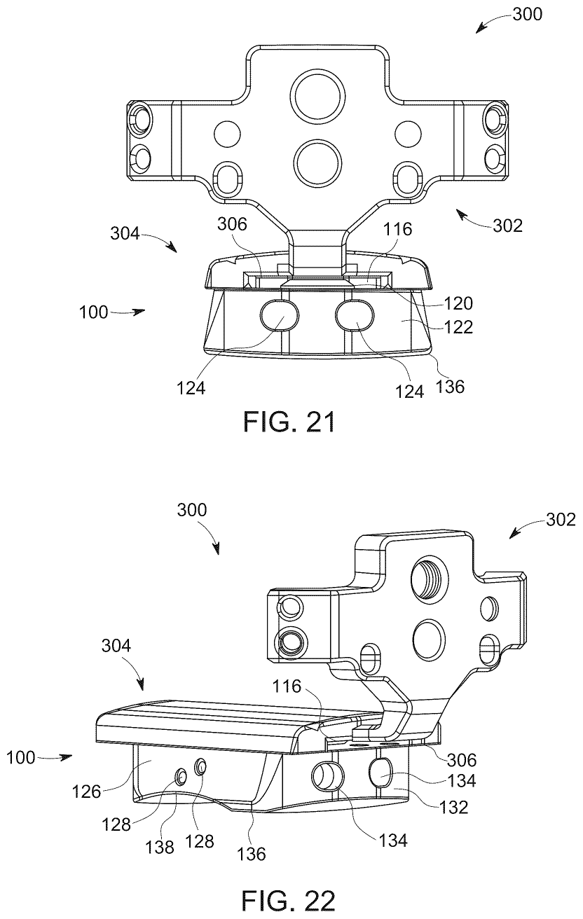

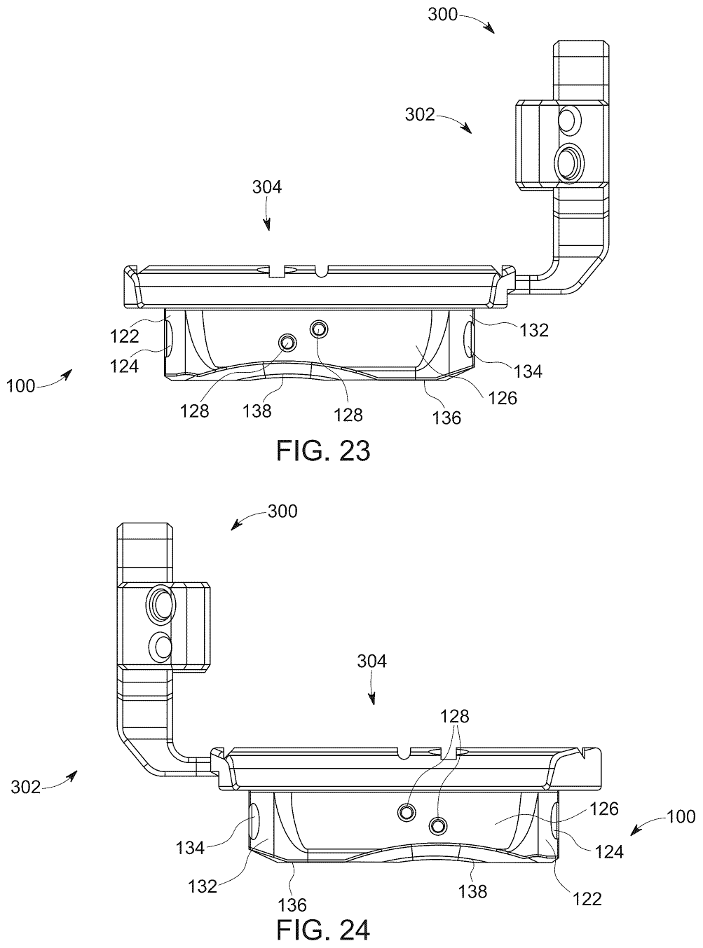

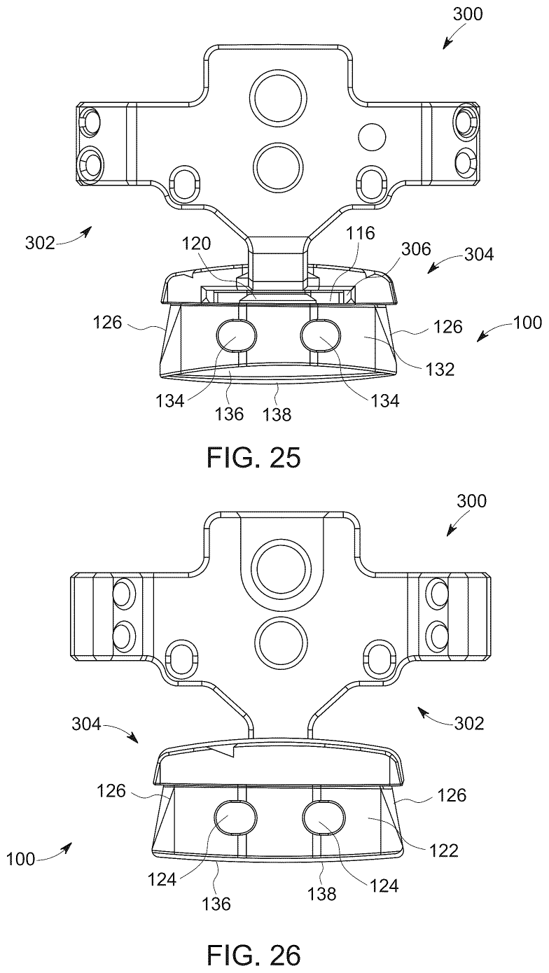

Referring now to FIGS. 18-26, an instrument 300 is shown to be releasably coupled with the trial 100. As shown, the instrument 300 is a tibial trial instrument which may include one or more of the same/similar features and/or geometries of the tibial component 200. The instrument 300 is shown to include an engagement portion 302 which may be configured to releasably couple with one or more other instruments configured to facilitate manipulation, positioning, and/or repositioning of the instrument 300 and any components releasably coupled therewith (e.g., the trial 100). In some aspects, the engagement portion 302 may include one or more holes or apertures configured to receive at least a portion of one or more protrusions of an instrument to facilitate releasable coupling. The instrument 300 is further shown to include a trial portion 304, where the trial portion 304 may have various geometric features and/or other components that are the same as and/or similar to a corresponding tibial implant component (e.g., tibial component 200). In some aspects, a physician may manipulate the instrument 300 such that the trial portion 304 is positioned within an area at or near that in which the physician may place a tibial implant component. Further, the physician may perform such manipulation of the instrument 300 with the trial 100 releasably coupled with the trial portion 304 via a slot 306 positioned on a bottom surface of the trial portion 304. In some aspects, the slot 306 may be the same as and/or similar to the recess 206 of the tibial component 210 and, further, the trial 100 may releasably couple therewith in the same and/or similar manner. (in anteriorly-biased and/or posteriorly-biased configurations). The instrument 300 may also be decoupled with the trial 100 and/or be releasably coupled with other trials of various sizes (in anteriorly-biased and/or posteriorly-biased configurations). Similarly, the trial 100 may be releasably coupled/decoupled with various sizes of the instrument 300.

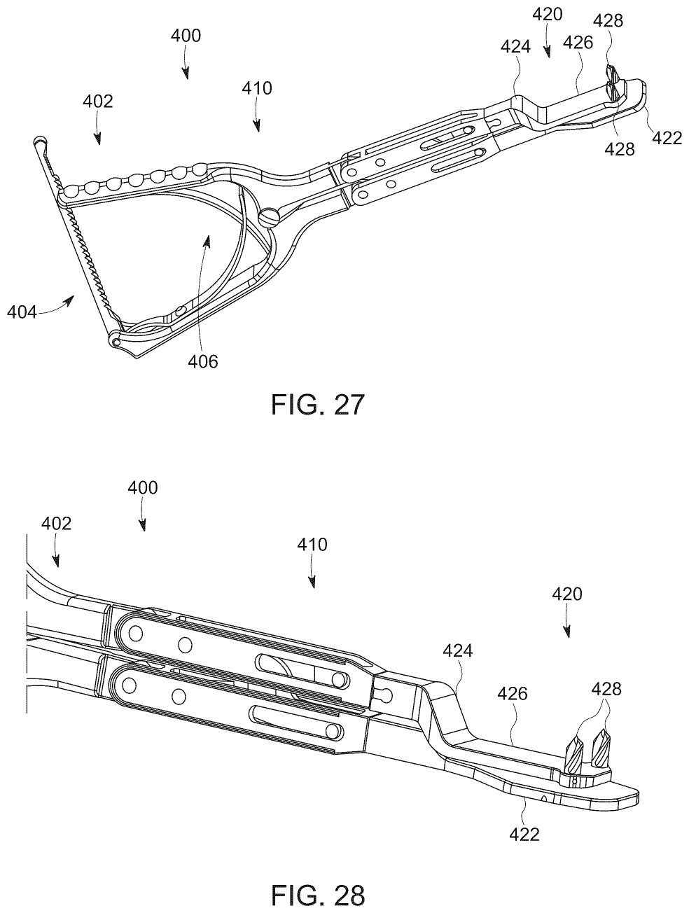

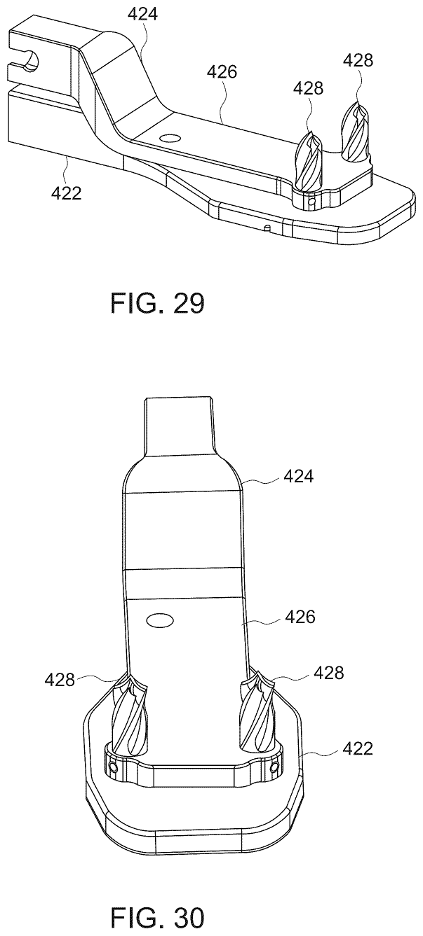

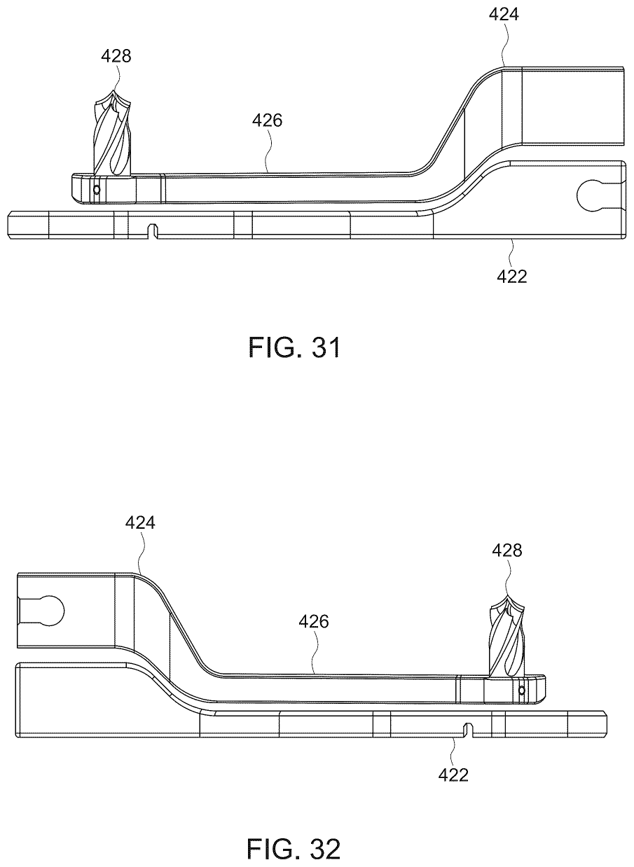

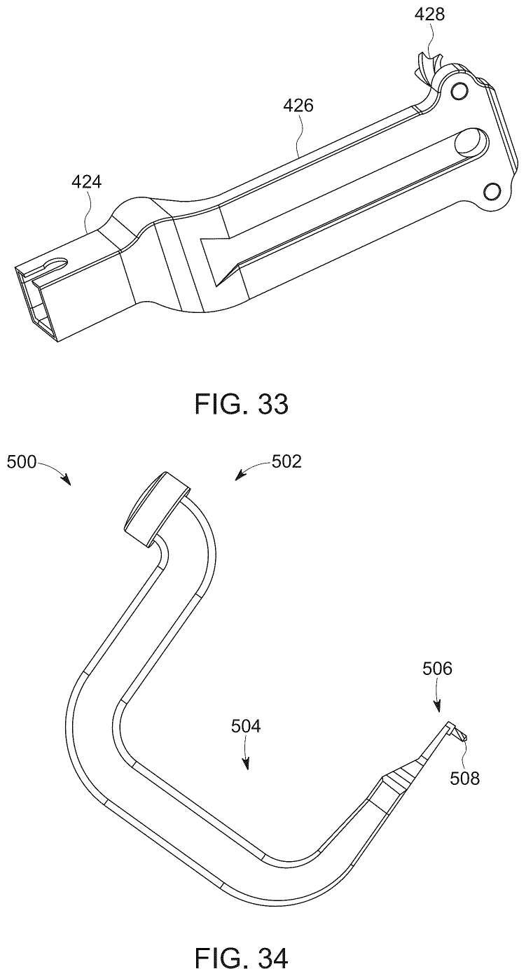

Referring now to FIGS. 27-33, an instrument 400 is shown, according to an exemplary embodiment. The instrument 400 is shown to include a handle 402 and an actuator 404 positioned adjacent the handle 402. The instrument 400 further includes a resilient member 406 disposed adjacent the handle 402 and, as shown, between two portions of the handle 402 such that the resilient member 406 may bias the portions of the handle 402 apart from one another. The actuator 404 may be actuated to retain the aforementioned portions of the handle 402 in a desired position despite any force(s) applied thereto by the resilient member 406. The instrument 400 is further shown to include a four-bar mechanism 410 (referred to herein as a four bar 410) that is shown to be coupled with the handle 402. The four bar 410 includes one or more bars (or, up to four bars) configured to expand and/or retract in response to the movement/actuation of the handle 402 in response to manipulation thereof and/or force(s) applied thereto by the resilient member 406.

The instrument 400 is further shown to include a punch 420 releasably coupled to a portion of the four bar 410 opposite the four bar 410 from the handle 402. The punch is shown to include an upper portion 424 configured to receive at least a portion of the four bar 410 therein so as to facilitate releasable coupling therewith. The punch 420 also includes a platform 426 positioned opposite the upper portion 424 from the four bar 410 where the platform 426 is integral with the upper portion 424. The punch further includes a pair of projections 428 positioned at a terminal end of the platform 426. In some aspects, the platform 426 may include a pair of protuberances at the terminal end thereof, with the projections 428 extending upward in a substantially perpendicular direction to the platform 426. The instrument 400 is also shown to include a paddle 422 configured to couple with the four bar 410 below the coupling point of the four bar 410 and the punch 420. The paddle 422 may be configured to contact a first surface opposite a second surface in which a user desired to create a volume/holes complimentary to the geometry of the projections 428. Accordingly, by manipulating the instrument 400, the paddle may contact the first surface so as to provide leverage as the punch 420 and projections 428 are manipulated upward to contact and create the aforementioned volume/holes in the second surface.

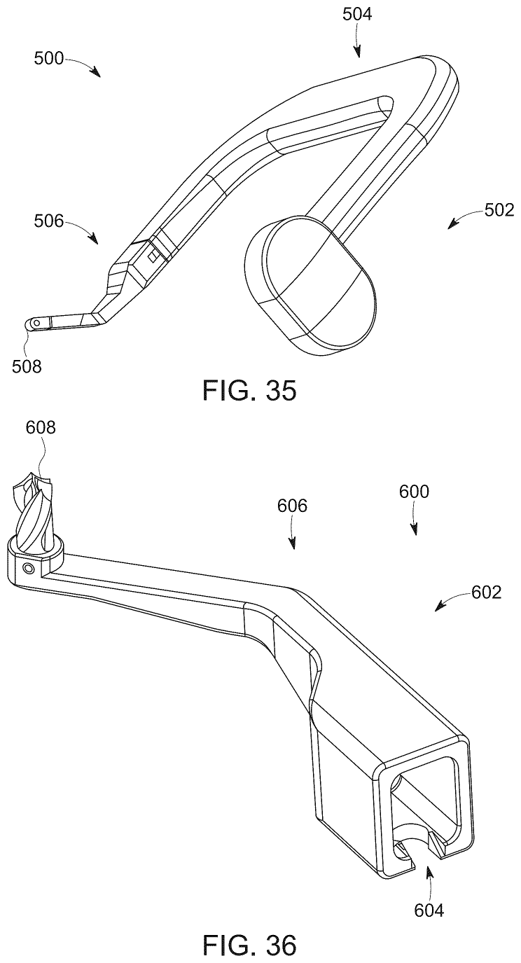

Referring now to FIGS. 34-35, an instrument 500 (shown as an impacting instrument, or an “impactor”) is shown, according to an exemplary embodiment. The instrument 500 is shown to include a body 504 separating an upper portion 502 and a lower portion 506. The upper portion 502 is configured to include an oblong geometry (which may have an alternate geometry in some aspects) at its terminal end, with the geometry configured to receive an impact force and translate the impact force mechanically about the instrument 500. For example, the instrument 500 may be placed in a desired position such that the lower portion 506 (and/or a component thereof, for example, a projection 508 extending from the lower portion 506) contacts or is adjacent a surface to which a user desires to apply a force/impact. Accordingly, an impact force may be applied to the oblong geometry in a plane that is substantially parallel to the protrusion 508 of the lower portion 506 such that the impact force is applied to the surface that is adjacent or contacting the protrusion 508. As shown, the body 504 is substantially perpendicular to and is integral with the upper and lower portions 502, 506, which are substantially parallel to one another.

Referring now to FIG. 36, another surgical instrument 600 is shown, according to an exemplary embodiment. The instrument 600 is shown to include an upper portion 602 integral with a lower portion 606. The upper portion 602 is shown to include an aperture 604 positioned cross-sectionally at a terminal end thereof and extending at least partially into the upper portion 602 so as to receive a component at least partially within the aperture 604 so as to facilitate releasable coupling. The lower portion 606 is shown to include a projection 608 extending substantially perpendicular from the lower portion 606, where the projection 608 may include one or more features thereon (e.g., threading, flutes, etc.) that are configured to facilitate punching a volume/hole in a portion of the anatomy where the volume/hole is complimentary to the geometry of the projection 608.

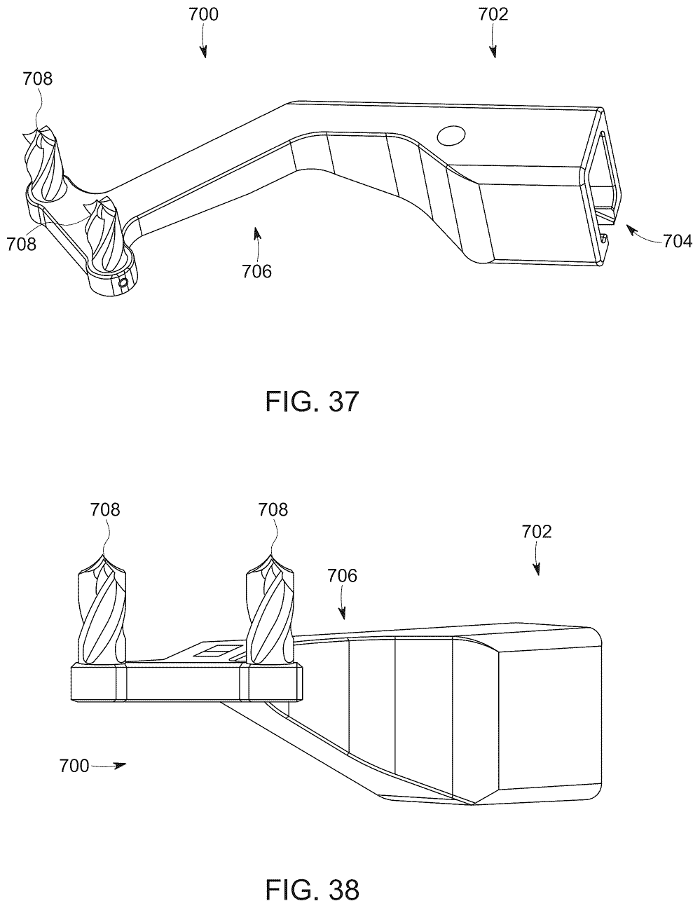

Referring now to FIGS. 37-38, another surgical instrument 700 is shown, according to an exemplary embodiment. The instrument 700 is shown to include an upper portion 702 integral with a lower portion 707. The upper portion 702 is shown to include an aperture 704 positioned cross-sectionally at a terminal end thereof and extending at least partially into the upper portion 702 so as to receive a component at least partially within the aperture 704 so as to facilitate releasable coupling. The lower portion 706 is shown to include at least one projection 708 extending in a substantially perpendicular direction from the lower portion 706, where the at least one projection 708 may include one or more features thereon (e.g., threading, flutes, etc.) configured to facilitate punching a volume/hole in a portion of the anatomy where the volume/hole is complimentary to the geometry of the at least one projection 708. As shown, the at least one projection 708 includes a pair of projections having the same and/or similar geometry. However, in some aspects the instrument 700 may include one or more projections 708 of various sizes, geometries, and positions about the instrument 700.

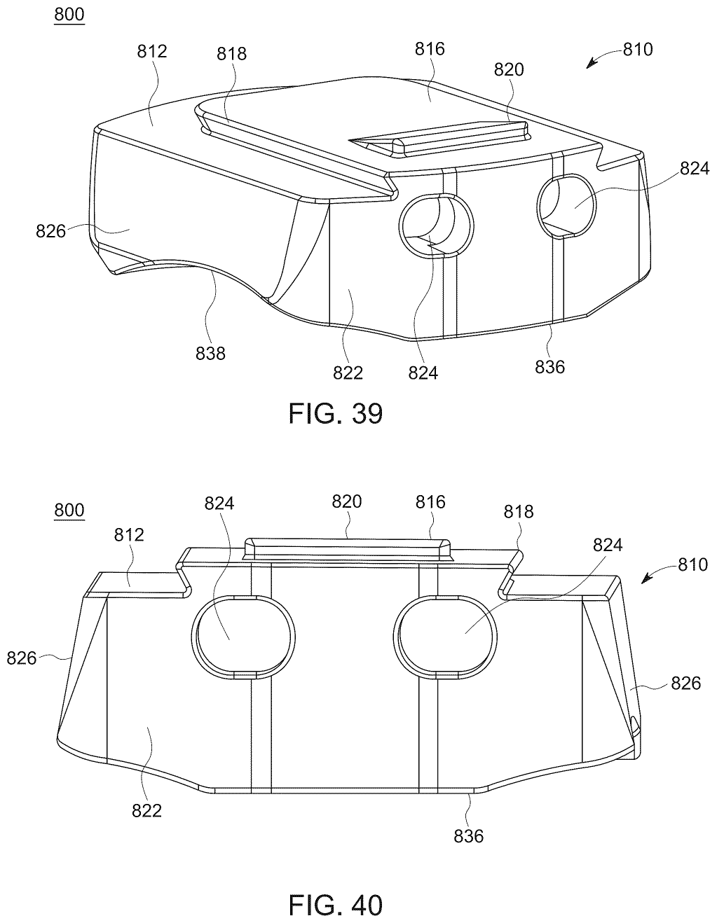

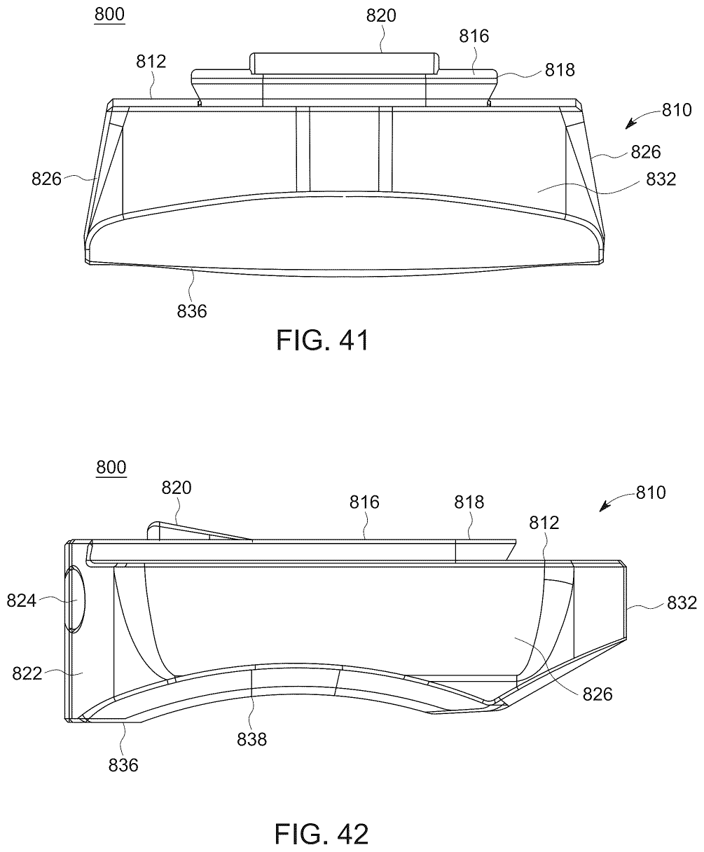

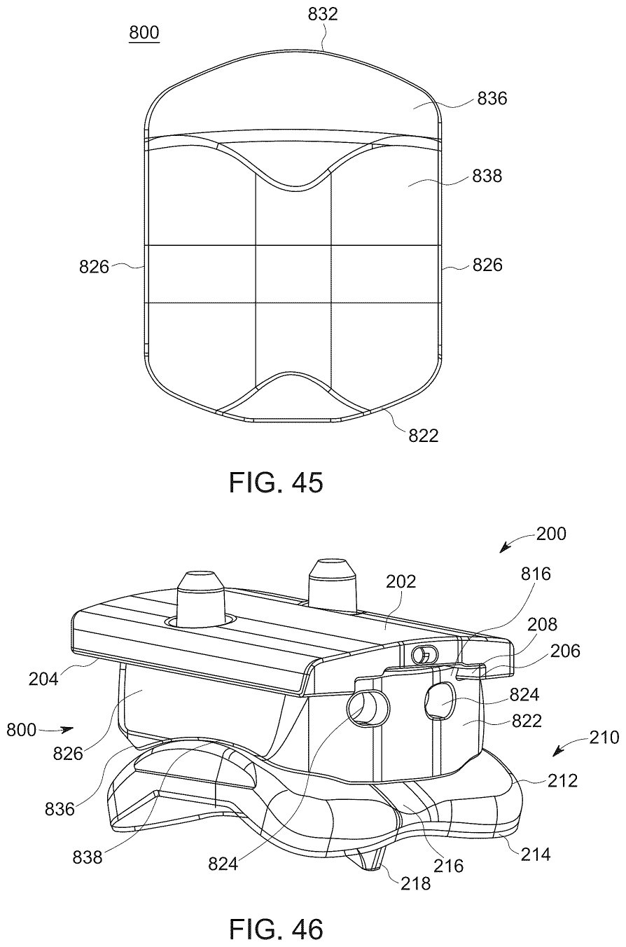

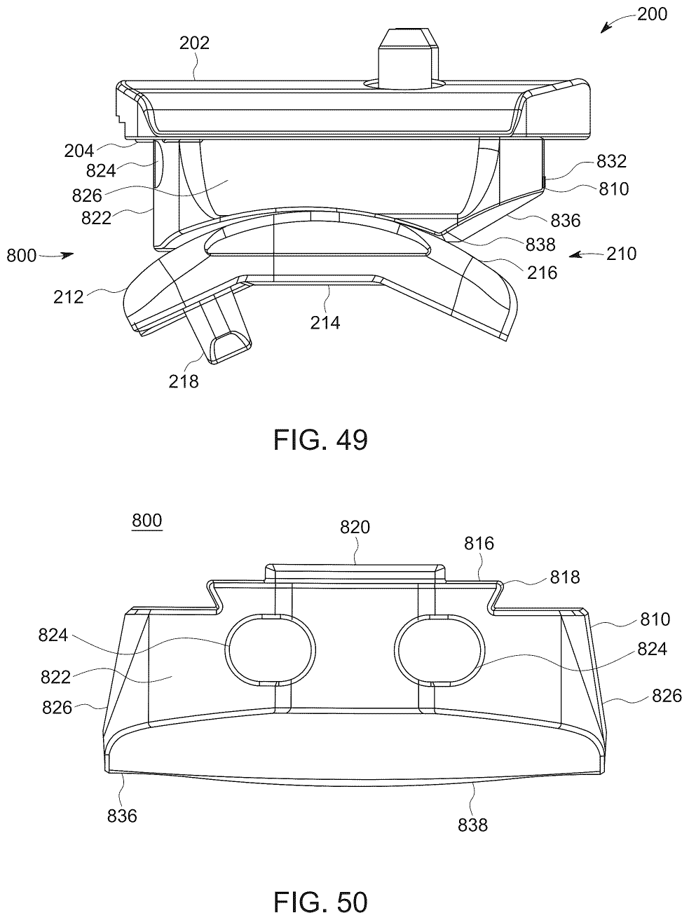

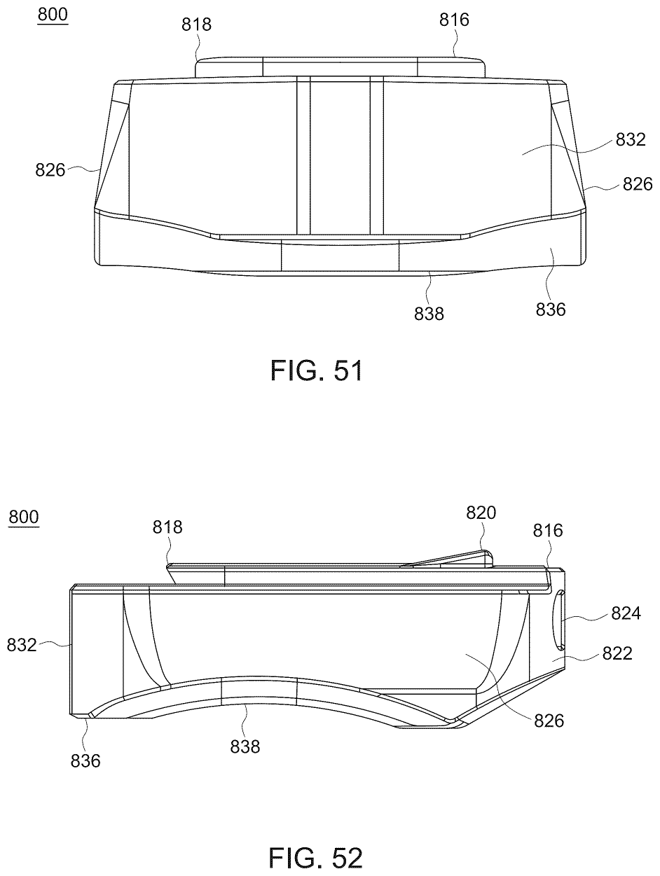

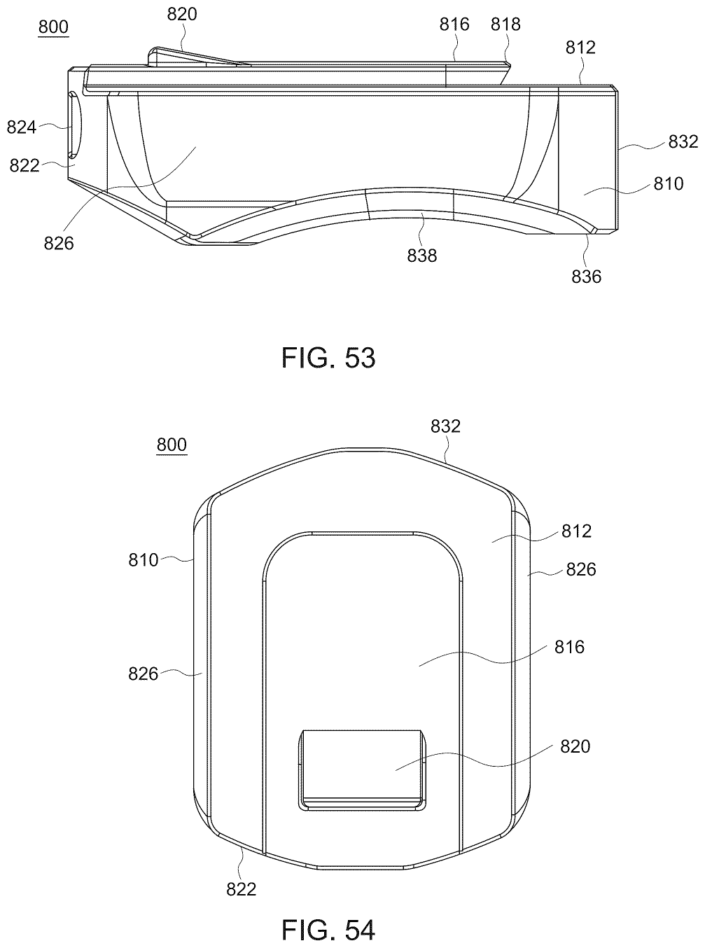

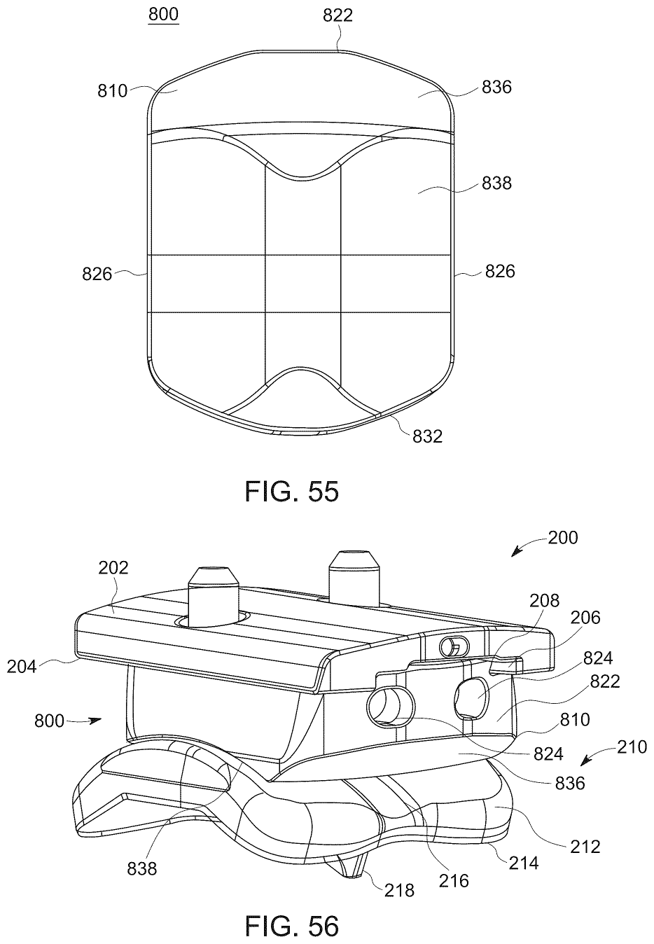

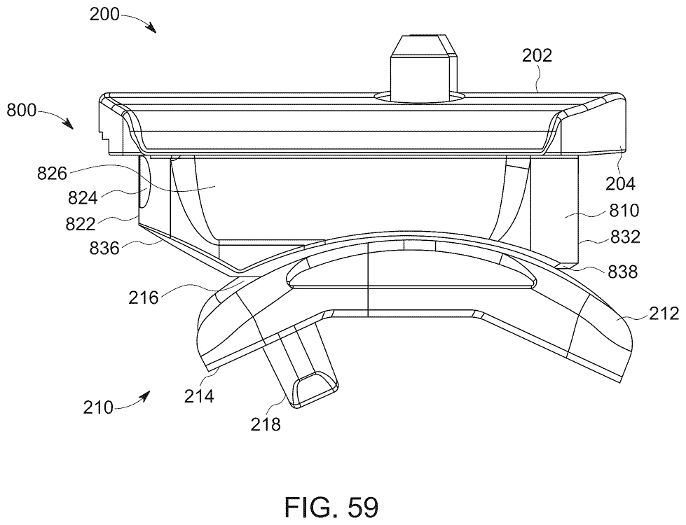

Referring now to FIGS. 39-59, an implant 800 is shown, according to one aspect of the present disclosure. The implant 800 is shown to include a body 810 including an upper surface 812 opposite a lower surface 836, a pair of lateral surfaces 826 opposite one another, and a front surface 822 opposite a rear surface 832. As shown, the body 810 has a substantially rectangular prism-based configuration, where the upper and lower surfaces 812, 836, the lateral surfaces 826, and the front and rear surfaces 822, 832 are substantially perpendicular to one another, respectively. The body 810 is shown to include a protrusion 816 extending in an upward direction from the upper surface 812, where the protrusion includes a ledge 818 extending at least partially around the perimeter of the protrusion 816 between the upper surface 812 of the body 810 and a top surface of the protrusion 816 that is substantially parallel to the upper surface 812. In some aspects, the protrusion 816 may abut the front surface 812 of the implant 800. In some aspects, the protrusion 816 and the ledge 818 thereof may collectively form a coupling means, for example, to facilitate releasable coupling with one or more other instruments and/or implant components. In some aspects, the implant 800 may be configured to releasably couple with surgical instruments and/or implants via the protrusion 816 and the ledge 818 in a dovetail configuration, where the protrusion 816 and the ledge 818 form a male dovetail component and are received in the instruments and/or implants by a corresponding female dovetail component. The male/female dovetail coupling arrangement is an example as other coupling mechanisms are contemplated. As shown, the protrusion 816 includes a notch 820 positioned on an upper surface of the protrusion 816 and adjacent to the front surface 822 of the implant 800, with the notch 820 centered relative to the lateral sides 826 of the implant 800 (with the protrusion 816 also being centered laterally relative to the top surface 812 of the implant 800). The notch 820 may be configured to facilitate coupling of the implant 800 with one more other components, either as a coupling feature and/or as a feature intended to limit insertion of the protrusion/ledge 816/818 into a corresponding volume of an instrument/implant component.

The front surface 822 (which is marked with at least one of “A” or “P” to indicate “anterior” and/or “posterior” bias to the implant, where the front surface 822 may be referred to as the anterior surface 822) is each shown to include a pair of apertures 824 disposed on the surface and extending into the body 810 of the implant 800 in a direction substantially perpendicular to the plane in which the front surface 822 is positioned. In some aspects, the apertures 824 may be configured to releasably couple the implant 800 with an instrument, for example an insertion instrument configured to facilitate positioning the implant 800 in a desired location as part of an implant trialing/size determination process, or other components of an implant/instrument kit/system. For example, one or more of the apertures 824 may be configured to receive a protrusion of an instrument and retain at least a portion of the protrusion therein while the implant 800 is manipulated, positioned, and/or repositioned.

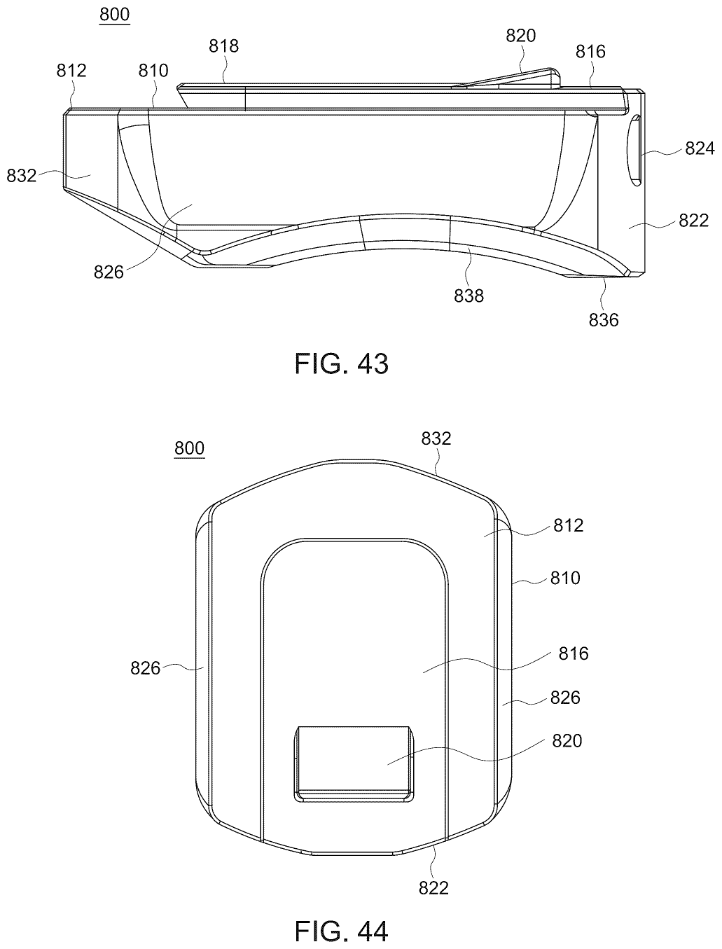

The implant 800 is further shown to include a concavity 838 positioned on/within at least a portion of the lower surface 836. The concavity 838 may have the same and/or similar geometry to the concavity 138 of the trial 100, just as the implant 800 may have one or more various geometric features/sizes/articulations which are the same as and/or similar to the trial 100. In some aspects, the concavity 838 may include a ridge along a central portion thereof separating two smaller sub-concavities. Further, in some aspects, the concavity 838 may correspond to a geometry of one or more implants and/or portions thereof with which the implant 800 is configured to interface in a trialing/size determining process. For example, the concavity 838 may correspond to a convexity/curvature of an upper surface of a talar implant with which the implant 800 is configured to interface. As shown in FIGS. 42-43, the concavity 838 is biased toward the front surface 822 of the implant 800. The implant 800 may be inserted such that the front surface 822 is facing the user and, accordingly, the concavity 838 may be positioned closer to the front surface 822 than the rear surface 832.

Referring now to FIGS. 46-49, the implant 800 is shown in conjunction with the implant system shown previously, including the tibial component 200 and the talar component 210. The tibial and talar components 200, 210 may be the same as and/or similar to other similar tibial and talar components of ankle arthroplasty systems, including but not limited to, those incorporated by reference herein. The recess 206 is configured to include the engagement feature 208 disposed therein which, as shown in at least FIGS. 46-49, includes a female dovetail configuration configured to interface and releasably couple with the protrusion 816, the ledge 818 of the implant 800, thus releasably coupling the implant 800 with the tibial component 200 (such that the tibial component 200 is positioned adjacent the notch 820). In some aspects, the implant 800 may be configured to engage/releasably couple with the tibial component 210 in the same manner as a trial component (e.g., the trial 100) to which the implant 800 corresponds (e.g., an intermediate trial component which may include the same/similar features to couple with the tibial component 200 in the same/similar manner). When engaged (e.g., releasably coupled) with the tibial component 200, the implant 800 is shown to be positioned substantially below (e.g., inferior, distal, etc.) relative to the tibial component 210).

The talar component 210 is shown to be positioned such that the implant 800 is disposed substantially above (e.g., superior, proximal, etc.) to the talar component 210. The top surface 212 and corresponding articulation 216 thereof are configured to engage/interface with the implant 800. The articulation 216 may be the same as and/or similar to that shown and described in the aforementioned issued patents/pending applications which have been incorporated by reference herein. For example, the articulation 216 may include the corresponding articulating surface being broken into four regions for reference, with the regions being anterior-medial, anterior-lateral, posterior-medial, and posterior-lateral (where the anterior portion of the talar component 210 is the portion that includes the engagement feature 218), with each region having at least one sagittal radius. In some aspects, the anterior-lateral sagittal radius may be greater than the anterior-medial sagittal radius, and the posterior-medial sagittal radius may be greater than the posterior-lateral sagittal radius. The concavity 838 of the implant 800 is configured to interface with the articulation 216 and, accordingly, may have a geometry that is complimentary to (e.g., a negative of) the articulation 216 and substantially the same and/or similar to the concavity of an implant component of which the implant 800 is representative.

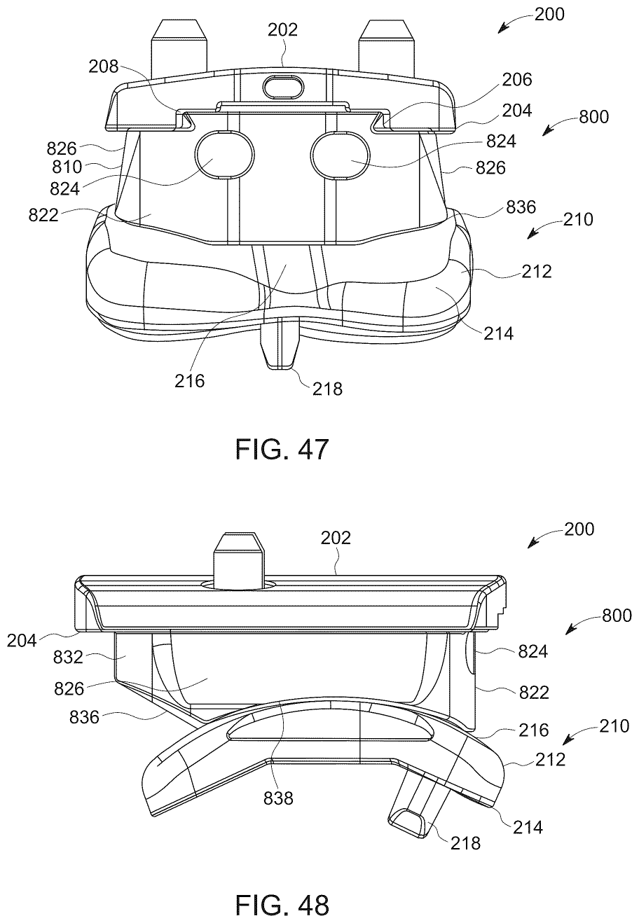

As shown in FIGS. 46-49, the implant 800 is shown (see at least the front view of FIG. 47) with the front surface 822 facing outward and adjacent the front (e.g., anterior) portions of the tibial and talar components 200, 210, with the “A” of the implant 800 indicating that the concavity 838 of the implant 800 is biased anteriorly (e.g., closer to the front/anterior surface 822). Contrary to the trial 100, if a physician were to determine that a switch from an anteriorly-biased implant 800 to a posteriorly-biased implant 800 were necessary, a different implant 800 would be required as the implant 800 does not have the reversibility of the trial 100. The implant 800, as shown in FIGS. 46-49 may be selected from multiple implants having the same configuration of the implant 800 as shown, but in various sizes where a trial such as the trial 100 has been implemented in the trialing process to determine a desired implant size and the physician has selected the implant 800 which corresponds to the desired size.

Referring now to FIGS. 50-55, the implant 800 is shown in an alternate, posteriorly-biased configuration with the “P” of the implant 800 indicating that the concavity 138 is biased posteriorly (e.g., closer to the rear/posterior surface 832). The implant 800 of FIGS. 50-55 may include some and/or all of the same features of the implant 800 of FIGS. 39-45, with the exception being the concavity 838 being biased such that it is closer to the rear/posterior surface 832 than the front/anterior surface 822. The trialing process for the implant 800 of FIGS. 50-55 may be the same as and/or similar to that described previously herein with reference to the implant 800 of FIGS. 39-45, although the trial 100 may be implemented in the reverse configuration (e.g., the posteriorly-biased trial configuration).

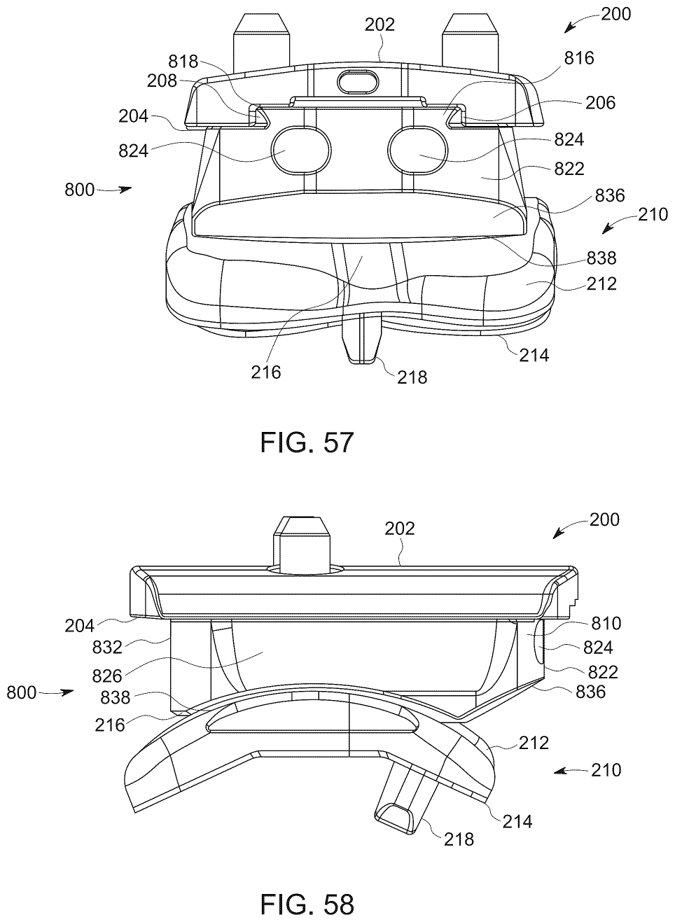

Referring now to FIGS. 56-59, the implant 800 is shown in a posteriorly-biased configuration (see at least the front view of FIG. 57) with the front surface 822 facing outward and the adjacent front (e.g., anterior) portions of the tibial and talar components 200, 210, with the “P” of the implant 800 indicating that the concavity 838 of the implant 800 is biased posteriorly (e.g., closer to the rear/posterior surface 832). Contrary to the trial 100, if a physician were to determine that a switch from posteriorly-biased implant 800 to an anteriorly-biased implant 800 were necessary, a different implant 800 would be required as the implant 800 does not have the reversibility of the trial 100. The implant 800 as shown in FIGS. 56-59 may be selected from multiple implants having the same configuration of the implant 800 as shown, but in various sizes where a trial such as the trial 100 has been implemented in a trialing process to determine a desired implant size and the physician has selected the implant 800 which corresponds to that desired size.

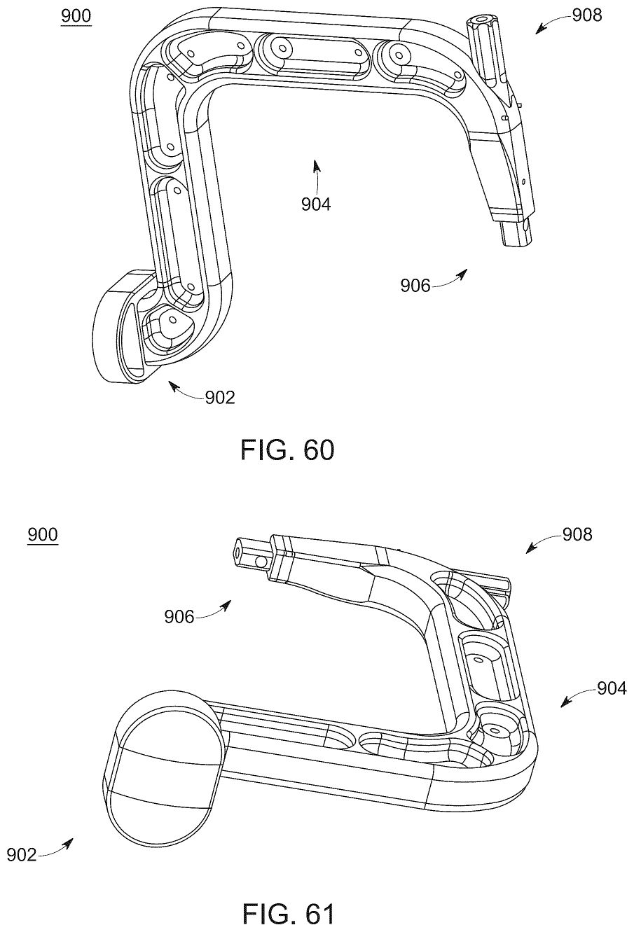

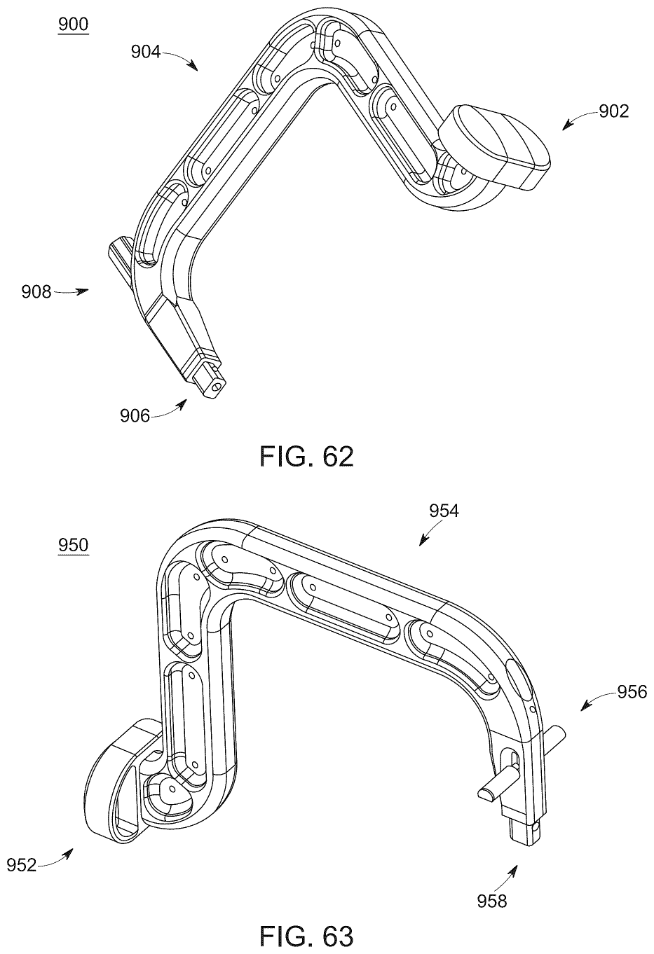

Referring now to FIGS. 60-62, an instrument 900 (shown as an impacting instrument, or an “impactor”) is shown, according to an exemplary embodiment. The instrument 900 is shown to include a body 904 separating an upper portion 902 and a lower portion 906. The upper portion 902 is configured to include an oblong geometry (which may have an alternate geometry in some aspects) at its terminal end, with the geometry configured to receive an impact force and translate the impact force mechanically about the instrument 900. For example, the instrument 900 may be placed in a desired position such that the lower portion 906 (and/or a component coupled therewith, for example via a coupling element 908) contacts or is adjacent a surface to which a user desires to apply a force/impact. The coupling element 908 may extend through a portion of the lower portion 906 and include an engagement feature (e.g., threading, a ball detent, etc.) that is actuatable to facilitate releasable coupling/decoupling with the instrument 900. Accordingly, an impact force may be applied to the oblong geometry in a plane that is substantially parallel to a protrusion of the lower portion 906 such that the impact force is applied to the surface that is adjacent or contacting the aforementioned protrusion. As shown, the body 904 is substantially perpendicular to and is integral with the upper and lower portions 902, 906, which are substantially parallel to one another.

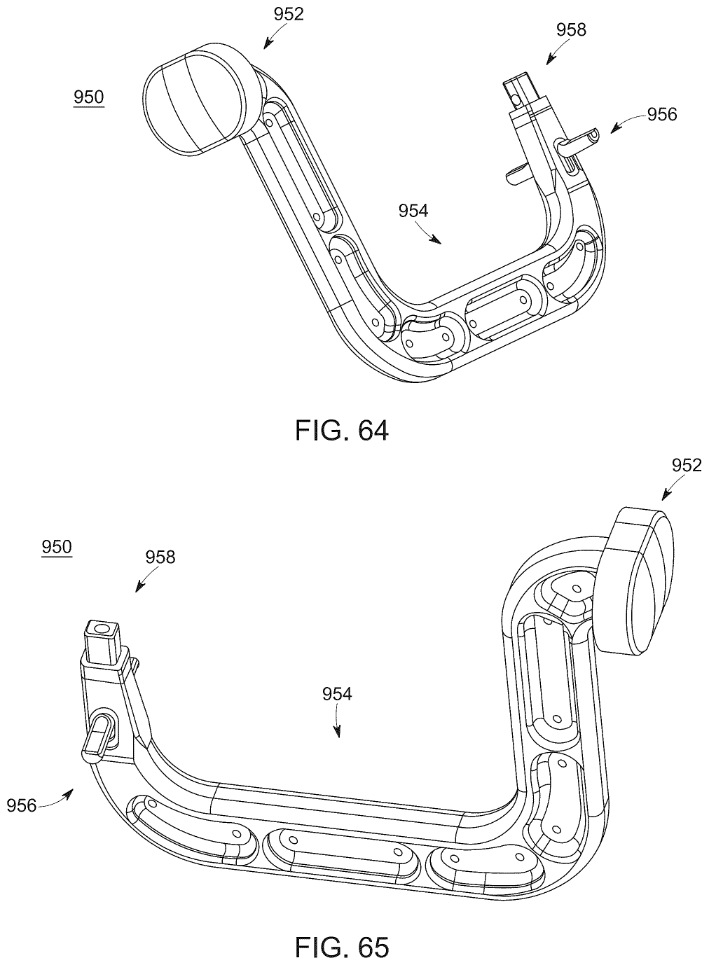

Referring now to FIGS. 63-65, an instrument 950 (shown as an impacting instrument, or an “impactor”) is shown, according to an exemplary embodiment. The instrument 950 is shown to include a body 954 separating an upper portion 952 and a lower portion 958. The upper portion 952 is configured to include an oblong geometry (which may have an alternate geometry in some aspects) at its terminal end, with the geometry configured to receive an impact force and translate the impact force mechanically about the instrument 950. For example, the instrument 950 may be placed in a desired position such that the lower portion 958 (and/or a component coupled therewith, for example via a coupling element 956) contacts or is adjacent a surface to which a user desires to apply a force/impact. The coupling element 956 may extend through a portion of the lower portion 958 and include an engagement feature (e.g., one or more of a threading, a ball detent, one or more laterally-protruding pegs, etc.) that may be actuatable to facilitate releasable coupling/decoupling with the instrument 950. Accordingly, an impact force may be applied to the oblong geometry in a plane that is substantially parallel to a protrusion of the lower portion 958 such that the impact force is applied to the surface that is adjacent or contacting the aforementioned protrusion. As shown, the body 954 is substantially perpendicular to and is integral with the upper and lower portions 952, 958, which are substantially parallel to one another.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

What is claimed:

1. An orthopedic implant, comprising:
a body comprising:
a top surface opposite a bottom surface;
a front surface opposite a rear surface; and
a pair of lateral sides positioned on opposite sides of the body from one another;
wherein at least a portion of the bottom surface comprises a concave geometry extending from one lateral side to the other lateral side, wherein the concave geometry is biased such that the concave geometry is either closer to the front surface or the rear surface of the body of the implant.

2. The orthopedic implant of claim 1, further comprising at least one aperture positioned on the front surface of the body of the implant.

3. The orthopedic implant of claim 2, wherein the at least one aperture comprises a pair of apertures positioned on opposite sides of a midline of the implant.

4. The orthopedic implant of claim 1, wherein the concave geometry is biased such that the concave geometry is closer to the front surface of the body of the implant.

5. The orthopedic implant of claim 1, wherein the concave geometry is biased such that the concave geometry is closer to the rear surface of the body of the implant.

6. The orthopedic implant of claim 1, further comprising a protrusion extending upward from the top surface of the body.

7. The orthopedic implant of claim 6, wherein the protrusion comprises a substantially rectangular geometry.

8. The orthopedic implant of claim 7, wherein the protrusion comprises a top surface that is spaced vertically from and in a plane substantially parallel to the top surface of the body of the implant.

9. The orthopedic implant of claim 9, wherein the protrusion comprises a lateral dimension that increases along the height of the protrusion from adjacent the top surface of the body of the implant to the top surface of the protrusion.

10. The orthopedic implant of claim 10, wherein the protrusion comprises a male dovetail component.

11. The orthopedic implant of claim 10, wherein the protrusion comprises a notch extending upward from the top surface of the protrusion, wherein the notch is centered laterally about the protrusion.

12. The orthopedic implant of claim 11, wherein the implant is couplable with another implant via the protrusion and notch.

13. An orthopedic implant component, comprising:
a body comprising:
a top surface opposite a bottom surface;
a front surface opposite a rear surface; and
a pair of lateral sides positioned on opposite sides of the body from one another;
wherein at least a portion of the bottom surface comprises a concave geometry extending from one lateral side to the other lateral side, and is positioned between the front and rear surfaces of the body.

14. The orthopedic implant component of claim 13, wherein the concave geometry is positioned closer to the front surface of the body than the rear surface of the body.

15. The orthopedic implant component of claim 14, wherein the concave geometry is positioned closer to the rear surface of the body than the front surface of the body.

16. The orthopedic implant of claim 13, wherein the implant comprises a protrusion extending upward from the top surface of the body of the implant component.

17. The orthopedic implant of claim 7, wherein the protrusion comprises a top surface that is spaced vertically from and in a plane substantially parallel to the top surface of the body of the implant.

18. The orthopedic implant of claim 9, wherein the protrusion comprises a lateral dimension that increases along the height of the protrusion from adjacent the top surface of the body of the implant to the top surface of the protrusion.

19. The orthopedic implant of claim 10, wherein the protrusion comprises a male dovetail component.

20. An orthopedic implant, comprising:
a body comprising:
a top surface opposite a bottom surface;
a front surface opposite a rear surface; and
a pair of lateral sides positioned on opposite sides of the body from one another;
wherein at least a portion of the bottom surface comprises a concave geometry extending from one lateral side to the other lateral side, wherein the concave geometry is biased such that the concave geometry is either closer to the front surface or the rear surface of the body of the implant; and
a protrusion extending upward from the top surface of the body and having a top surface in a plane substantially parallel to the top surface of the body, wherein the protrusion comprises a lateral dimension that increases along the height of the protrusion from a position adjacent the top surface of the body to the top surface of the protrusion.
